SEQUENCE LISTING (62 sequences)

SEQ ID NO: 1 - protein, 112 aa, Artificial Sequence
  CD3 Z endodomain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 2 - protein, 152 aa, Artificial Sequence
  CD28 and CD3 Zeta endodomains
SKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 3 - protein, 188 aa, Artificial Sequence
  CD28, OX40 and CD3 Zeta endodomains
SKRSRLLHSDYMNMTPRRPGPTRKHYQPYAPPRDFAAYRSRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 4 - protein, 341 aa, Artificial Sequence
  CD148 endodomain
RKKRKDAKNNEVSFSQIKPKKSKLIRVENFEAYFKKQQADSNCGFAEEYEDLKLVGISQPKYAAELAENRGKNRYNNVLPYDISRVKLSVQTHSTDDYINANYMPGYHSKKDFIATQGPLPNTLKDFWRMVWEKNVYAIIMLTKCVEQGRTKCEEYWPSKQAQDYGDITVAMTSEIVLPEWTIRDFTVKNIQTSESHPLRQFHFTSWPDHGVPDTTDLLINFRYLVRDYMKQSPPESPILVHCSAGVGRTGTFIAIDRLIYQIENENTVDVYGIVYDLRMHRPLMVQTEDQYVFLNQCVLDIVRSQKDSKVDLIYQNTTAMTIYENLAPVTTFGKTNGYIA

SEQ ID NO: 5 - protein, 707 aa, Artificial Sequence
  CD45 endodomain
KIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 6 - protein, 352 aa, Artificial Sequence
  SHP-1 phosphatase domain
FWEEFESLQKQEVKNLHQRLEGQRPENKGKNRYKNILPFDHSRVILQGRDSNIPGSDYINANYIKNQLLGPDENAKTYIASQGCLEATVNDFWQMAWQENSRVIVMTTREVEKGRNKCVPYWPEVGMQRAYGPYSVTNCGEHDTTEYKLRTLQVSPLDNGDLIREIWHYQYLSWPDHGVPSEPGGVLSFLDQINQRQESLPHAGPIIVHCSAGIGRTGTIIVIDMLMENISTKGLDCDIDIQKTIQMVRAQRSGMVQTEAQYKFIYVAIAQFIETTKKKLEVLQSQKGQESEYGNITYPPAMKNAHAKASRTSSKHKEDVYENLHTKNKREEKVKKQRSADKEKSKGSLKRK

SEQ ID NO: 7 - protein, 351 aa, Artificial Sequence
  SHP-2 phosphatase domain
FWEEFETLQQQECKLLYSRKEGQRQENKNKNRYKNILPFDHTRVVLHDGDPNEPVSDYINANIIMPEFETKCNNSKPKKSYIATQGCLQNTVNDFWRMVFQENSRVIVMTTKEVERGKSKCVKYWPDEYALKEYGVMRVRNVKESAAHDYTLRELKLSKVGQALLQGNTERTVWQYHFRTWPDHGVPSDPGGVLDFLEEVHHKQESIVDAGPVVVHCSAGIGRTGTFIVIDILIDIIREKGVDCDIDVPKTIQMVRSQRSGMVQTEAQYRFIYMAVQHYIETLQRRIEEEQKSKRKGHEYTNIKYSLVDQTSGDQSPLPPCTPTPPCAEMREDSARVYENVGLMQQQRSFR

SEQ ID NO: 8 - protein, 38 aa, Artificial Sequence
  ICOS endodomain
CWLTKKKYSSSVHDPNGEYMFMRAVNTAKKSRLTDVTL

SEQ ID NO: 9 - protein, 48 aa, Artificial Sequence
  CD27 endodomain
QRRKYRSNKGESPVEPAEPCHYSCPREEEGSTIPIQEDYRKPEPACSP

SEQ ID NO: 10 - protein, 111 aa, Artificial Sequence
  BTLA endodomain
RRHQGKQNELSDTAGREINLVDAHLKSEQTEASTRQNSQVLLSETGIYDNDPDLCFRMQEGSEVYSNPCLEENKPGIVYASLNHSVIGPNSRLARNVKEAPTEYASICVRS

SEQ ID NO: 11 - protein, 188 aa, Artificial Sequence
  CD30 endodomain
HRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 12 - protein, 58 aa, Artificial Sequence
  GITR endodomain
QLGLHIWQLRSQCMWPRETQLLLEVPPSTEDARSCQFPEEERGERSAEEKGRLGDLWV

SEQ ID NO: 13 - protein, 60 aa, Artificial Sequence
  HVEM endodomain
CVKRRKPRGDVVKVIVSVQRKRQEAEGEATVIEALQAPPDVTTVAVEETIPSFTGRSPNH

SEQ ID NO: 14 - protein, 97 aa, Artificial Sequence
  PD1 endodomain
CSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 15 - protein, 97 aa, Artificial Sequence; identical to SEQ ID NO: 14
  PDCD1 endodomain

SEQ ID NO: 16 - protein, 141 aa, Artificial Sequence
  BTLA4 endodomain
KLQRRWKRTQSQQGLQENSSGQSFFVRNKKVRRAPLSEGPHSLGCYNPMMEDGISYTTLRFPEMNIPRTGDAESSEMQRPPPDCDDTVTYSALHKRQVGDYENVIPDFPEDEGIHYSELIQFGVGERPQAQENVDYVILKH

SEQ ID NO: 17 - protein, 168 aa, Artificial Sequence
  LILRB1 endodomain
LRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 18 - protein, 101 aa, Artificial Sequence
  LAIR1 endodomain
HRQNQIKQGPPRSKDEEQKPQQRPDLAVDVLERTADKATVNGLPEKDRETDTSALAAGSSQEVTYAQLDHWALTQRTARAVSPQSTKPMAESITYAAVARH

SEQ ID NO: 19 - protein, 62 aa, Artificial Sequence
  CTLA4 endodomain
FLLWILAAVSSGLFFYSFLLTAVSLSKMLKKRSPLTTGVYVKMPPTEPECEKQFQPYFIPIN

SEQ ID NO: 20 - protein, 111 aa, Artificial Sequence
  KIR2DL1 endodomain
GNSRHLHVLIGTSVVIIPFAILLFFLLHRWCANKKNAVVMDQEPAGNRTVNREDSDEQDPQEVTYTQLNHCVFTQRKITRPSQRPKTPPTDIIVYTELPNAESRSKVVSCP

SEQ ID NO: 21 - protein, 143 aa, Artificial Sequence
  KIR2DL4 endodomain
GIARHLHAVIRYSVAIILFTILPFFLLHRWCSKKKENAAVMNQEPAGHRTVNREDSDEQDPQEVTYAQLDHCIFTQRKITGPSQRSKRPSTDTSVCIELPNAEPRALSPAHEHHSQALMGSSRETTALSQTQLASSNVPAAGI

SEQ ID NO: 22 - protein, 143 aa, Artificial Sequence
  KIR2DL5 endodomain
TGIRRHLHILIGTSVAIILFIILFFFLLHCCCSNKKNAAVMDQEPAGDRTVNREDSDDQDPQEVTYAQLDHCVFTQTKITSPSQRPKTPPTDTTMYMELPNAKPRSLSPAHKHHSQALRGSSRETTALSQNRVASSHVPAAGI

SEQ ID NO: 23 - protein, 111 aa, Artificial Sequence
  KIR3DL1 endodomain
KDPRHLHILIGTSVVIILFILLLFFLLHLWCSNKKNAAVMDQEPAGNRTANSEDSDEQDPEEVTYAQLDHCVFTQRKITRPSQRPKTPPTDTILYTELPNAKPRSKVVSCP

SEQ ID NO: 24 - protein, 97 aa, Artificial Sequence
  KIR3DL3 endodomain
KDPGNSRHLHVLIGTSVVIIPFAILLFFLLHRWCANKKNAVVMDQEPAGNRTVNREDSDEQDPQEVTYAQLNHCVFTQRKITRPSQRPKTPPTDTSV

SEQ ID NO: 25 - protein, 449 aa, Homo sapiens
  MISC_FEATURE: full length C-terminal Src kinase (CSK)
SAIQAAWPSGTECIAKYNFHGTAEQDLPFCKGDVLTIVAVTKDPNWYKAKNKVGREGIIPANYVQKREGVKAGTKLSLMPWFHGKITREQAERLLYPPETGLFLVRESTNYPGDYTLCVSCDGKVEHYRIMYHASKLSIDEEVYFENLMQLVEHYTSDADGLCTRLIKPKVMEGTVAAQDEFYRSGWALNMKELKLLQTIGKGEFGDVMLGDYRGNKVAVKCIKNDATAQAFLAEASVMTQLRHSNLVQLLGVIVEEKGGLYIVTEYMAKGSLVDYLRSRGRSVLGGDCLLKFSLDVCEAMEYLEGNNFVHRDLAARNVLVSEDNVAKVSDFGLTKEASSTQDTGKLPVKWTAPEALREKKFSTKSDVWSFGILLWEIYSFGRVPYPRIPLKDVVPRVEKGYKMDAPDGCPPAVYEVMKNCWHLDAAMRPSFLQLREQLEHIKTHELHL

SEQ ID NO: 26 - protein, 256 aa, Artificial Sequence
  sequence of tyrosine kinase domain of CSK
LKLLQTIGKGEFGDVMLGDYRGNKVAVKCIKNDATAQAFLAEASVMTQLRHSNLVQLLGVIVEEKGGLYIVTEYMAKGSLVDYLRSRGRSVLGGDCLLKFSLDVCEAMEYLEGNNFVHRDLAARNVLVSEDNVAKVSDFGLTKEASSTQDTGKLPVKWTAPEALREKKFSTKSDVWSFGILLWEIYSFGRVPYPRIPLKDVVPRVEKGYKMDAPDGCPPAVYEVMKNCWHLDAAMRPSFLQLREQLEHIKTHELHL

SEQ ID NO: 27 - protein, 7 aa, Artificial Sequence
  Tobacco Etch Virus (TeV) cleavage recognition site
ENLYFQS

SEQ ID NO: 28 - protein, 240 aa, Artificial Sequence
  TeV protease domain
SLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQTKSMSSMVSDTSCTFPSSDGIFWKHWIQTKDGQCGSPLVSTRDGFIVGIHSASNFTNTNNYFTSVPKNFMELLTNQEAQQWVSGWRLNADSVLWGGHKVFMSKPEEPFQPVKEATQLMNELVYSQ

SEQ ID NO: 29 - protein, 108 aa, Artificial Sequence
  FKBP12 dimerization domain
MGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 30 - protein, 100 aa, Artificial Sequence
  wild-type FKBP-rapamycin binding (FRB) segment of mTOR
MASRILWHEMWHEGLEEASRLYFGERNVKGMFEVLEPLHAMMERGPQTLKETSFNQAYGRDLMEAQEWCRKYMKSGNVKDLTQAWDLYYHVFRRISKLES

SEQ ID NO: 31 - protein, 100 aa, Artificial Sequence
  FRB with T to L substitution at 2098 which allows binding to AP21967
MASRILWHEMWHEGLEEASRLYFGERNVKGMFEVLEPLHAMMERGPQTLKETSFNQAYGRDLMEAQEWCRKYMKSGNVKDLLQAWDLYYHVFRRISKLES

SEQ ID NO: 32 - protein, 100 aa, Artificial Sequence
  FRB segment of mTOR with T to H substitution at 2098 and W to F at residue 2101of full mTOR
MASRILWHEMWHEGLEEASRLYFGERNVKGMFEVLEPLHAMMERGPQTLKETSFNQAYGRDLMEAQEWCRKYMKSGNVKDLHQAFDLYYHVFRRISKLES

SEQ ID NO: 33 - protein, 100 aa, Artificial Sequence
  FRB segment of mTOR with K to P substitution at residue 2095 of full mTOR
MASRILWHEMWHEGLEEASRLYFGERNVKGMFEVLEPLHAMMERGPQTLKETSFNQAYGRDLMEAQEWCRKYMKSGNVPDLTQAWDLYYHVFRRISKLES

SEQ ID NO: 34 - protein, 139 aa, Artificial Sequence
  Tet repressor (TetR) sequence
MSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGH

SEQ ID NO: 35 - protein, 17 aa, Artificial Sequence
  TetR interacting protein (TiP) sequence
MWTWNAYAFAAPSGGGS

SEQ ID NO: 36 - protein, 15 aa, Artificial Sequence
  Biotin mimicking peptide, long nanotag
DVEAWLDERVPLVET

SEQ ID NO: 37 - protein, 9 aa, Artificial Sequence
  Biotin mimicking peptide, short nanotag
DVEAWLGAR

SEQ ID NO: 38 - protein, 8 aa, Artificial Sequence
  Biotin mimicking peptide, streptag
WRHPQFGG

SEQ ID NO: 39 - protein, 8 aa, Artificial Sequence
  Biotin mimicking peptide, streptagII
WSHPQFEK

SEQ ID NO: 40 - protein, 38 aa, Artificial Sequence
  Biotin mimicking peptide, SBP-tag
MDEKTTGWRGGHVVEGLAGELEQLRARLEHHPQGQREP

SEQ ID NO: 41 - protein, 8 aa, Artificial Sequence
  Biotin mimicking peptide, ccstreptag
CHPQGPPC

SEQ ID NO: 42 - protein, 15 aa, Artificial Sequence
  Biotin mimicking peptide, flankedccstreptag
AECHPQGPPCIEGRK

SEQ ID NO: 43 - protein, 126 aa, Artificial Sequence
  core streptavidin sequence
EAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAAS

SEQ ID NO: 44 - protein, 20 aa, Foot-and-mouth disease virus
RAEGRGSLLTCGDVEENPGP

SEQ ID NO: 45 - protein, 20 aa, Foot-and-mouth disease virus
QCTNYALLKLAGDVESNPGP

SEQ ID NO: 46 - protein, 20 aa, Artificial Sequence
  signal peptide SFGmR
METDTLLLWVLLLWVPGSTG

SEQ ID NO: 47 - protein, 23 aa, Artificial Sequence
  Linker-V5 tag-Linker sequence
DSSGKPIPNPLLGLDSSGGGGSA

SEQ ID NO: 48 - protein, 183 aa, Artificial Sequence
  CD22(2Ig) membrane proximal Ig domains
PRDVRVRKIKPLSEIHSGNSVSLQCDFSSSHPKEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPYHSQKLRLEPVKVQHSGAYWCQGTNSVGKGRSPLSTLTVYYSPETIGRR

SEQ ID NO: 49 - protein, 39 aa, Artificial Sequence
  CD19 transmembrane sequence and truncated CD19 endodomain, CD19tm
AVTLAYLIFCLCSLVGILHLQRALVLRRKRKRMTDPTRR

SEQ ID NO: 50 - protein, 58 aa, Artificial Sequence
  Rigid Linker sequence, RL
LEAEAAAKEAAAKEAAAKEAAAKALEAEAAAKEAAAKEAAAKEAAAKALESGGGSASR

SEQ ID NO: 51 - protein, 99 aa, Artificial Sequence
  FRB domain
ILWHEMWHEGLEEASRLYFGERNVKGMFEVLEPLHAMMERGPQTLKETSFNQAYGRDLMEAQEWCRKYMKSGNVKDLLQAWDLYYHVFRRISKLEYSAS

SEQ ID NO: 52 - protein, 18 aa, Artificial Sequence
  Foot-and-Mouth disease virus (FMDV) 2A peptide
EGRGSLLTCGDVEENPGP

SEQ ID NO: 53 - protein, 107 aa, Artificial Sequence
  FKBP12 domain sequence, FKBP12
GVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 54 - protein, 12 aa, Artificial Sequence
  TetR interacting peptide, TIP
MWTWNAYAFAAP

SEQ ID NO: 55 - protein, 16 aa, Artificial Sequence
  linker sequence
SGGGGSGGGGSGGGGS

SEQ ID NO: 56 - protein, 207 aa, Artificial Sequence
  Tet repressor B protein, TetRB
MSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGS

SEQ ID NO: 57 - protein, 99 aa, Artificial Sequence
  destabilisation domain, FK506-rapamycin binding (FRB) FRBmut
ASRILWHEMWHEGLEEASRLYFGERNVKGMFEVLEPLHAMMERGPQTLKETSFNQAYGRDLMEAQEWCRKYMKSGNVPDLLQAFDLYYHVFRRISKLEY

SEQ ID NO: 58 - protein, 107 aa, Artificial Sequence
  destabilisation domain, mutant of the FKBP12 F36V sequence
GVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLE

SEQ ID NO: 59 - protein, 107 aa, Artificial Sequence
  destabilisation domain, FKBP12 L106P
GVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKPE

SEQ ID NO: 60 - protein, 498 aa, Artificial Sequence
  second generation anti-CD19 CAR sequence
METDTLLLWVLLLWVPGSTGDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITKAGGGGSGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSDPTTTPAPRPPTPAPTIASQPLSLRPEACRPAAGGAVHTRGLDFACDIYIWAPLAGTCGVLLLSLVITLYCRKKRSRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 61 - protein, 6 aa, Artificial Sequence
  linker sequence
SGGGSG

SEQ ID NO: 62 - protein, 6 aa, Artificial Sequence
  ITIM conserved sequence
  MISC_FEATURE (1)..(1): Xaa may be Ser, Ile, Val or Leu
  misc_feature (2)..(2): Xaa can be any naturally occurring amino acid
  misc_feature (4)..(5): Xaa can be any naturally occurring amino acid
  MISC_FEATURE (6)..(6): Xaa may be Ile, Val or Leu
XXYXXX

(19) United States
(12) Patent Application Publication
(10) Pub. No.: US 2020/0188434 A1
(43) Pub. Date: Jun. 18, 2020

(54) A CELL COMPRISING A CHIMERIC ANTIGEN RECEPTOR (CAR)

(71) Applicant: AUTOLUS LIMITED, London (GB)

(72) Inventors: Shaun Cordoba, London (GB); Evangelia Kokalaki, London (GB); Martin Pulé, London (GB); Simon Thomas, London (GB); Shimobi Onuoha, London (GB)

(21) Appl. No.: 16/613,343

(22) PCT Filed: May 14, 2018
(86) PCT No.: PCT/GB18/51293

§ 371 Date: Nov. 13, 2019

(30) Foreign Application Priority Data
May 15, 2017 (GB) 1707780.1
May 15, 2017 (GB) 1707781.9
May 15, 2017 (GB) 1707782.7

(51) Int. Cl. A61K 35/17; C07K 14/415; C07K 14/725; C07K 14/705

(52) U.S. Cl. CPC A61K 35/17; C07K 14/415; C07K 14/7051; C07K 2317/24; C07K 14/70589; C07K 2319/03; C07K 14/7056

(57) ABSTRACT

The present invention provides a cell which comprises; (i) a chimeric antigen receptor (CAR) which comprises an antigen binding domain and an intracellular signalling domain; (ii) a membrane tethering component (MTC) which comprises a first dimerization domain; and (Hi) a signal-dampening component (SDC) comprising a signal-dampening domain (SDD) and a second dimerization domain which specifically binds the first dimerisation domain of the membrane-tethering component. Dimerisation between the MTC and SDC may be controllable with an agent, meaning that the agent can be used to control CAR-mediated cell signalling.

### FIELD OF THE INVENTION

The present invention relates to a cell which comprises a chimeric antigen receptor (CAR).

### BACKGROUND TO THE INVENTION

Traditionally, antigen-specific T-cells have been generated by selective expansion of peripheral blood T-cells natively specific for the target antigen. However, it is difficult and quite often impossible to select and expand large numbers of T-cells specific for most cancer antigens. Gene-therapy with integrating vectors affords a solution to this problem as transgenic expression of Chimeric Antigen Receptor (CAR) allows generation of large numbers of T cells specific to any surface antigen by ex vivo viral vector transduction of a bulk population of peripheral blood T-cells.

Chimeric antigen receptors are proteins which graft the specificity of a monoclonal antibody (mAb) to the effector function of a T-cell. Their usual form is that of a type I transmembrane domain protein with an antigen recognizing amino terminus, a spacer, a transmembrane domain all connected to a compound endodomain which transmits T-cell survival and activation signals (see FIG. 1A).

The most common forms of these molecules are fusions of single-chain variable fragments (scFv) derived from monoclonal antibodies which recognize a target antigen, fused via a spacer and a trans-membrane domain to a signalling endodomain. Such molecules result in activation of the T-cell in response to recognition by the scFv of its target. When T cells express such a CAR, they recognize and kill target cells that express the target antigen. Several CARs have been developed against tumour associated antigens, and adoptive transfer approaches using such CAR-expressing T cells are currently in clinical trial for the treatment of various cancers.

A number of toxicities have been reported from CAR studies, and additional theoretical toxicities exist. Such toxicities include immunological toxicity caused by sustained intense activation of the CAR T-cells resulting in a macrophage activation syndrome (MAS) and “On-target off-tumour” toxicity i.e. recognition of the target antigen on normal tissues.

MAS is presumed to be caused by persistent antigen-driven activation and proliferation of T-cells which in turn release copious inflammatory cytokines leading to hyper-activation of macrophages and a feed-forward cycle of immune activation. A large spike in serum IL-6 is characteristic and the syndrome can result in a severe systemic illness requiring ICU admission.

On-target off-tumour toxicity has been reported with other CARs, for example a group of patients treated with a CAR against the renal cell carcinoma antigen CAIX developed unexpected and treatment limiting biliary toxicity. Two fatalities have been reported with CAR studies: one patient died of a respiratory distress syndrome which occurred immediately post-infusion of a large dose of 3rd generation anti-ERBB2 CAR T-cells; a further patient died in a different study after a possible cytokine storm following treatment of CLL with a second generation anti-CD19 CAR.

These toxicities are very difficult to predict even with detailed animal studies or non-human primate work. Crucially, unlike small molecules and biologics, CAR T-cells do not have a half-life and one cannot cease administration and wait for the agent to breakdown/become excreted. CAR T-cells are autonomous and can engraft and proliferate. Toxicity can therefore be progressive and fulminant.

Suicide genes are genetically expressed elements which can conditionally destroy cells which express them. Examples include Herpes-simplex virus thymidine kinase, which renders cells susceptible to Ganciclovir; inducible Caspase 9, which renders cells susceptible to a small molecular homodimerizer and CD20 and RQR8, which renders cells susceptible to Rituximab.

This technology adds a certain amount of safety to CAR T-cell therapy, however there are limitations. Firstly, it is a binary approach wherein all the CAR T-cells are destroyed upon addition of the suicide agent. In addition, medicinal therapeutics often have a therapeutic window. With a suicide gene the potency of the product cannot be tuned such that efficacy with tolerable toxicity can be achieved. Secondly, it is not clear whether a suicide gene would help with some of the immune-toxicities described above: for instance by the time a macrophage activation syndrome has been triggered, it may well no longer need the CAR T-cells to perpetuate and the suicide gene would no longer be helpful. The more acute cytokine release syndromes probably occur too quickly for the suicide gene to work.

There is therefore a need for alternative mechanisms to control CAR T cells, which are not associated with the disadvantages mentioned above.

### DESCRIPTION OF THE FIGURES

FIG. 1—a) Schematic diagram illustrating a classical CAR. (b) to (d): Different generations and permutations of CAR endodomains: (b) initial designs transmitted ITAM signals alone through FcεR1-γ or CD3ζ endodomain, while later designs transmitted additional (c) one or (d) two co-stimulatory signals in the same compound endodomain.

FIG. 2—Schematic diagram of a CAR signalling system which is made up of a CAR, a membrane tethering component (MTC) and a signal dampening component (SDC). In this example, the CAR comprises: an antigen-binding domain based on A Proliferation-Inducing Ligand (APRIL); a hinge spacer; a CD28 transmembrane domain; CD28 and OX40 co-stimulatory domains and a CD3 zeta endodomain (FIG. 2A). The MTC has: an extracellular domain comprising a V5 tag and spacer which is Ig domains 5 and 6 from CD22; a CD19 transmembrane domain; an intracellular linker, and a first dimerization domain which comprises FRB. The SDC comprises CD148 or CSK kinase as signal-dampening domain (SDD), together with FKBP12 as the second dimerization domain (FIG. 2B).

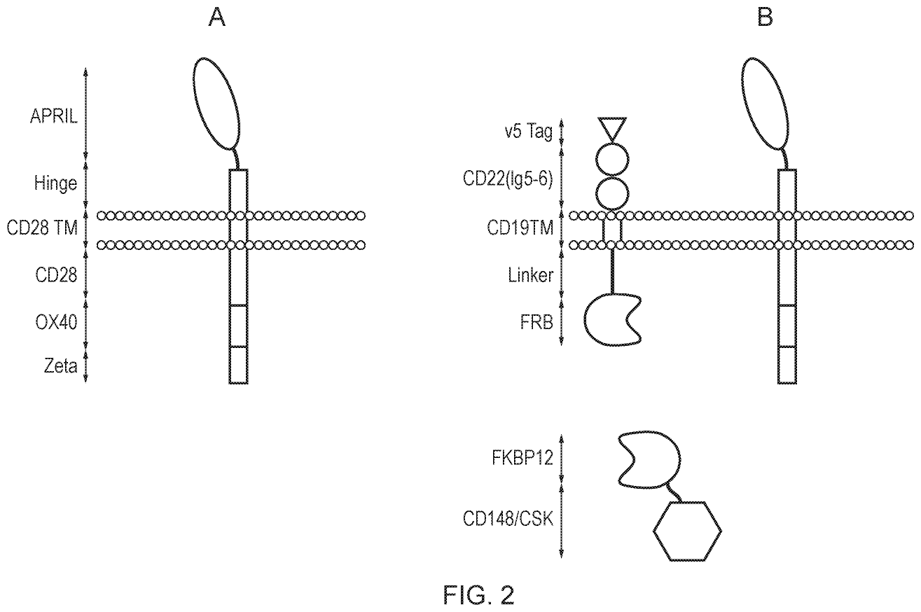

FIG. 3—Schematic diagram illustrating agent-mediated control of the CAR signalling system illustrated in FIG. 2. In the absence of rapamycin, the SDC moves freely inside the cell and does not affect CAR-mediated cell signalling (left-hand box). In the presence of rapamycin, FRB and FKBP12 dimerize, bringing the SDC to the cell membrane, where the SDD dampens CAR-mediated cell signalling (right-hand box).

FIG. 4—Schematic diagram of alternative CAR signalling systems, in which the dampening effect of the SDD is removed by the addition of an agent. The CAR is the same as the one shown in FIG. 2 (see A). In the arrangement shown in B, the MTC comprises: a myristoylation sequence; an intracellular linker, and a first dimerization domain which comprises TetRB; and the SDC comprises CD148 or CSK kinase as signal-dampening domain (SDD), together with Tet-interacting peptide (TiP) as the second dimerization domain. In the arrangement shown in C, the MTC comprises: an extracellular domain comprising a V5 tag and spacer which is Ig domains 5 and 6 from CD22; a CD19 transmembrane domain; an intracellular linker, and a first dimerization domain which comprises TetRB; and the SDC comprises CD148 or CSK kinase as signal-dampening domain (SDD), together with TiP as the second dimerization domain.

FIG. 5—Schematic diagram illustrating agent-mediated control of the CAR signalling system illustrated in FIG. 4B. In the absence of tetracycline, TetRB and TiP dimerize and the SDC is tethered to the cell membrane, where the SDD dampens CAR-mediated cell signalling signalling (left-hand box). In the presence of tetracycline, Tet out-competes TiP for binding to TetRB, so the SDC dissociates from the MTC such that the SDC does not affect CAR-mediated cell (right-hand box).

FIG. 6—Schematic diagram illustrating agent-mediated control of the CAR signalling system illustrated in FIG. 40. In the absence of tetracycline, TetRB and TiP dimerize and the SDC is tethered to the cell membrane, where the SDD dampens CAR-mediated cell signalling signalling (left-hand box). In the presence of tetracycline, Tet out-competes TiP for binding to TetRB, so the SDC dissociates from the MTC such that the SDC does not affect CAR-mediated cell (right-hand box).

FIG. 7(a)—Diagram of immediate T-cell activation pathways. T-cell receptor activation results in phosphorylation of ITAMs. Phosphorylated ITAMs are recognized by the ZAP70 SH2 domains. Upon recognition, ZAP70 is recruited to the juxta-membrane region and its kinase domain subsequently phosphorylates LAT. Phosphorylated LAT is subsequently recognized by the SH2 domains of GRAP, GRB2 and PLC-□. (b)—Diagram of immediate T-cell inhibition pathways. Activation of an inhibitory immune-receptor such as PD1 results in phosphorylation of ITIM domains. These are recognized by the SH2 domains of PTPN6. Upon recognition, PTPN6 is recruited to the juxta-membrane region and its phosphatase domain subsequently de-phosphorylates ITAM domains inhibiting immune activation.

FIG. 8—Schematic diagram of a CAR signalling system which is made up of a CAR and a signal dampening component (SDC). In this example, the CAR comprises: an antigen-binding domain (scFv); a CD8 spacer; a CD28 transmembrane domain; a first dimerization domain which comprises FRB; a CD28 co-stimulatory domains and a CD3 zeta endodomain (FIG. 8B). The equivalent “classical” CAR (FIG. 8A) lacks the FRB first dimerization domain. The SDC comprises CD148 or CSK kinase as signal-dampening domain (SDD), together with FKBP12 as the second dimerization domain (FIG. 8B).

FIG. 9—Schematic diagram illustrating agent-mediated control of the CAR signalling system illustrated in FIG. 8. In the absence of rapamycin, the SDC moves freely inside the cell and does not affect CAR-mediated cell signalling (left-hand box). In the presence of rapamycin, FRB and FKBP12 dimerize, bringing the SDC to the cell membrane, where the SDD dampens CAR-mediated cell signalling (right-hand box).

FIG. 10—Schematic diagram of an alternative CAR signalling system, in which the dampening effect of the SDD is removed by the addition of an agent. The CAR is the same as the one shown in FIG. 2 except that the first dimerization domain. comprises TetRB. (FIG. 10B). The equivalent “classical” CAR (FIG. 10A) lacks the TetRB first dimerization domain. The SDC comprises CD148 or CSK kinase as signal-dampening domain (SDD), together with Tet-interacting peptide (TiP) as the second dimerization domain.

FIG. 11—Schematic diagram illustrating agent-mediated control of the CAR signalling system illustrated in FIG. 10. In the absence of tetracycline, TetRB and TiP dimerize and the SDD dampens CAR-mediated cell signalling signalling (left-hand box). In the presence of tetracycline, Tet out-competes TiP for binding to TetRB, so the SDC dissociates from the CAR such that the SDC does not affect CAR-mediated cell (right-hand box).

FIG. 12—Schematic diagram of a CAR signalling system which is made up of a chimeric antigen receptor (CAR) and a membrane-tethered signal-dampening component (SDC) comprising a signal-dampening domain (SDD) and a destabilisation domain. In this example, the SDC has an ectodomain comprising two Ig domains from CD22; a transmembrane domain; a signal dampening (i.e. inhibitory) domain comprising CD148 kinase and a destamilisation domain comprising the mutant form of FRB (FRBmut). In the absence of rapamycin, the SDC is unstable and is either not expressed or degraded. CAR mediated cell signalling is therefore unaffected. In the presence of rapamycin, the SDC is stabilized and CD148 dampens CAR-mediated cell signalling by dephosphorylating ITAMs in the CAR endodomain.

FIG. 13—A: Results of an Incucyte assay comparing killing of BCMA+ SKOV3 target cells over time in the presence of varying concentrations of rapamycin by T cells expressing an anti-BCMA CAR alone (Left-hand chart) or expressing an anti-BCMA CAR in combination with a membrane tethering component (v5-CD22(2Ig)-TM-FRB) and a signal dampening component (FKBP12-CD148). In the presence of rapamycin, killing of target cells by T cells expressing a CAR, membrane tethering component and signal dampeneing component was significantly inhibited. The inhibition was titratable depending on the concentration of rapamycin.

B: A summary of the data presented in A at the 72 hour time-point. Killing of target cells by T cells expressing a CAR, membrane tethering component and signal dampeneing component was significantly inhibited at the tested concentrations of Rapamycin above 0.82 μM. Again, inhibition is shown to eb titratable with the concentration of Rapamycin.

FIG. 14—Results of an Incucyte assay comparing killing of CD19+ SKOV3 target cells over time in the absence of Rapamycin (red line) or presence of 10 μM Rapamycin (black line) with T cells expressing an anti-CD19 CAR alone (Left-hand chart) or expressing an anti-CD19 CAR in combination with a membrane tethering component (v5-CD22(2Ig)-TM-FRB) and a signal dampening component (FKBP12-CD148). In the presence of rapamycin, killing of target cells by T cells expressing a CAR, membrane tethering component and signal dampening component was significantly inhibited. At 50 hours, the control CAR had almost completely killed the target cells, whereas for T-cells co-expressing the CAR with a dampener, approximately 50% of the target cells were surviving.

### SUMMARY OF ASPECTS OF THE INVENTION

The present inventors have developed a CAR signalling system which is controllable with an agent. The signalling system comprises a signal dampening domain which inhibits CAR-mediated cell signalling. This means that CAR-mediated cell signalling can be turned down (or up) by administration of the agent, providing a mechanism for control, for example in the event of a CAR-associated toxicity. In a first aspect, the invention provides a cell which comprises;

(i) a chimeric antigen receptor (CAR) which comprises an antigen binding domain and an intracellular signalling domain;

(ii) a membrane tethering component which comprises a first dimerization domain; and

(iii) a signal-dampening component (SDC) comprising a signal-dampening domain (SDD) and a second dimerization domain which specifically binds the first dimerisation domain of the membrane-tethering component.

The SDD may inhibit the intracellular signalling domain of the CAR.

The SDD may comprise a phosphatase domain capable of dephosphorylating immunoreceptor tyrosine-based activation motifs (ITAMs).

The SDD may comprise the endodomain of CD148 or CD45.

The SDD may comprise the phosphatase domain of SHP-1 or SHP-2

The SDD may comprise an immunoreceptor tyrosine-based inhibition motif (ITIM).

The SDD may comprise an endodomain from one of the following inhibitory receptors: PD1, BTLA, 2B4, CTLA-4, GP49B, Lair-1, Pir-B, PECAM-1, CD22, Siglec 7, Siglec 9, KLRG1, ILT2, CD94-NKG2A and CD5.

The SDD may inhibit a Src protein kinase.

The SDD may inhibit Lck.

The SDD may comprise the kinase domain of CSK.

The SDD may cause the removal of the intracellular signalling domain of the CAR.

For example, the SDD may comprise a protease and the CAR may comprise a protease cleavage site.

The SDD may comprise Tobacco Etch Virus Protease (TeV).

Binding of the first and second dimerization domains may be controllable by the presence or absence of an agent.

In a first embodiment, binding of the first and second dimerization domains may be induced by the presence of a chemical inducer of dimerisation (CID).

In this respect, one dimerization domain may comprise an FK506-binding protein (FKBP), the other dimerization domain may comprise an FRB domain of mTOR and the CID may be rapamycin or a rapamycin analogue.

Alternatively, the first and second dimerization domains may comprise a FK506-binding protein (FKBP) and the CID may be FK1012.

Alternatively, the first and second dimerization domains may comprise GyrB and the CID may be coumermycin or a derivative thereof.

Alternatively, one dimerization domain may comprise GAI, the other dimerization domain may comprise GID1 and the CID may be gibberellin or a derivative thereof.

In a second embodiment, the presence of an agent disrupts binding of the first and second dimerization domains

In this respect, one dimerization domain may comprise the Tet repressor (TetR), the other dimerization domain may comprise TetR interacting protein (TiP) and the agent may be tetracycline, doxycycline, minocycline or an analogue thereof.

The membrane tethering component may comprise a transmembrane domain or a myristoylation sequence.

In a second aspect, the invention provides a nucleic acid construct which comprises:
(i) a first nucleic acid sequence which encodes a chimeric antigen receptor (CAR) as defined in the first aspect of the invention;
(ii) a second nucleic acid sequence which encodes a membrane-tethering component (MTC) as defined in the first aspect of the invention; and
(iii) a third nucleic acid sequence which encodes a signal-dampening component (SDC) as defined in the first aspect of the invention.

In a third aspect, the invention provides a kit of nucleic acid sequences comprising:
(i) a first nucleic acid sequence which encodes a chimeric antigen receptor (CAR) as defined in the first aspect of the invention;
(ii) a second nucleic acid sequence which encodes a membrane-tethering component (MTC) as defined in the first aspect of the invention; and
(ii) a third nucleic acid sequence which encodes a signal-dampening component (SDC) as defined in the first aspect of the invention.

In a fourth aspect, the invention provides a vector comprising a nucleic acid construct according to the second aspect of the invention.

In a fifth aspect, the invention provides a kit of vectors which comprises:
(i) a first vector which comprises a nucleic acid sequence which encodes a chimeric antigen receptor (CAR) as defined in the first aspect of the invention;
(ii) a second vector which comprises a nucleic acid sequence which encodes a membrane-tethering component (MTC) as defined in the first aspect of the invention; and
(ii) a third vector which comprises a nucleic acid sequence which encodes a signal-dampening component (SDC) as defined in the first aspect of the invention.

In a sixth aspect the invention provides a pharmaceutical composition comprising a plurality of cells according to the first aspect of the invention.

In a seventh aspect, the invention provides a pharmaceutical composition according to the sixth aspect of the invention for use in treating and/or preventing a disease.

In a seventh aspect, there is provided a method for treating and/or preventing a disease, which comprises the step of administering a pharmaceutical composition according to the sixth aspect of the invention to a subject.

The method may comprise the following steps:
(i) isolation of a cell-containing sample;
(ii) transduction or transfection of the cells with a nucleic acid construct according to the second aspect of the invention, a kit of nucleic acid sequences according to the third aspect of the invention; a vector according to the fourth aspect of the invention or a kit of vectors according to the fifth aspect of the invention; and
(iii) administering the cells from (ii) to a subject.

In an eighth aspect, the invention provides a method for controlling the activation of a cell according to the first aspect of the invention in a subject, which comprises the step of administering an agent which controls binding or dissociation of the first and second dimerization domains to the subject.

In a ninth aspect, the invention provides a method for treating a CAR-associated toxicity in a subject comprising a cell according to the first aspect of the invention, which comprises the step of administering an agent which induces binding of the first and second binding domains to the subject.

The CAR-associated toxicity may, for example, be cytokine release syndrome, macrophage activation syndrome, or a neurotoxicity.

In a tenth aspect, the invention provides the use of a pharmaceutical composition according to the first aspect of the invention in the manufacture of a medicament for the treatment and/or prevention of a disease.

The disease may be cancer.

In an eleventh aspect, the invention provides a method for making a cell according to the first aspect of the invention, which comprises the step of introducing a nucleic acid construct according to the second aspect of the invention, a kit of nucleic acid sequences according to the third aspect of the invention; a vector according to the fourth aspect of the invention, or a kit of vectors according to the fifth aspect of the invention into a cell.

The cell may be from a sample isolated from a subject.

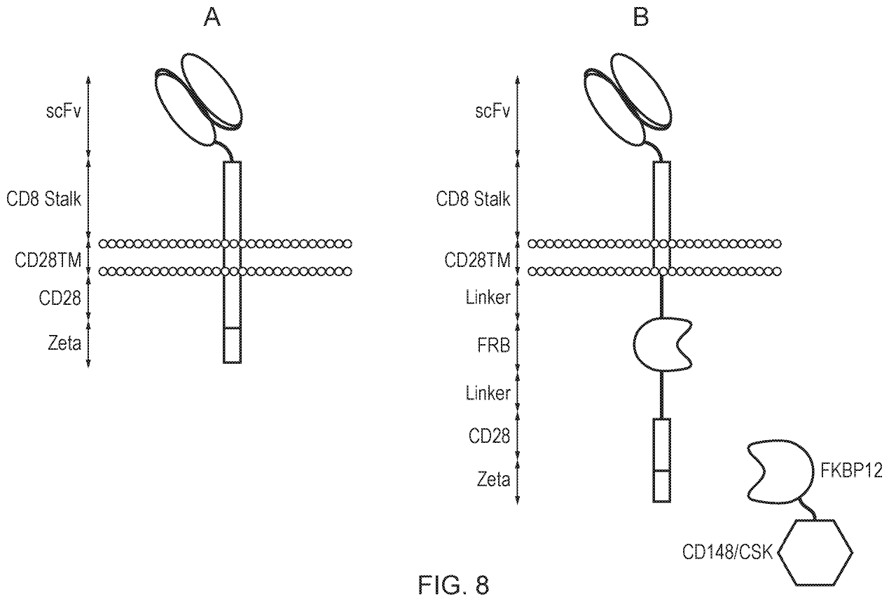

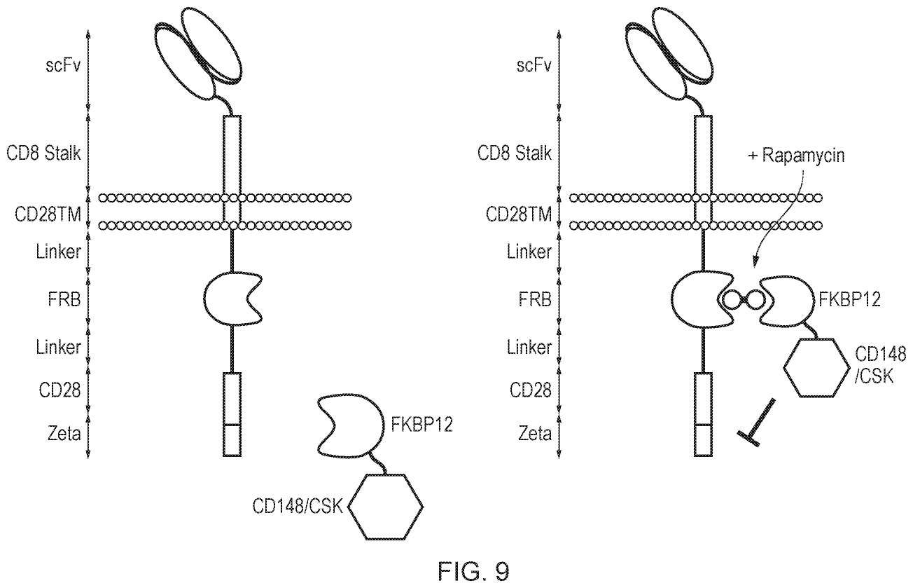

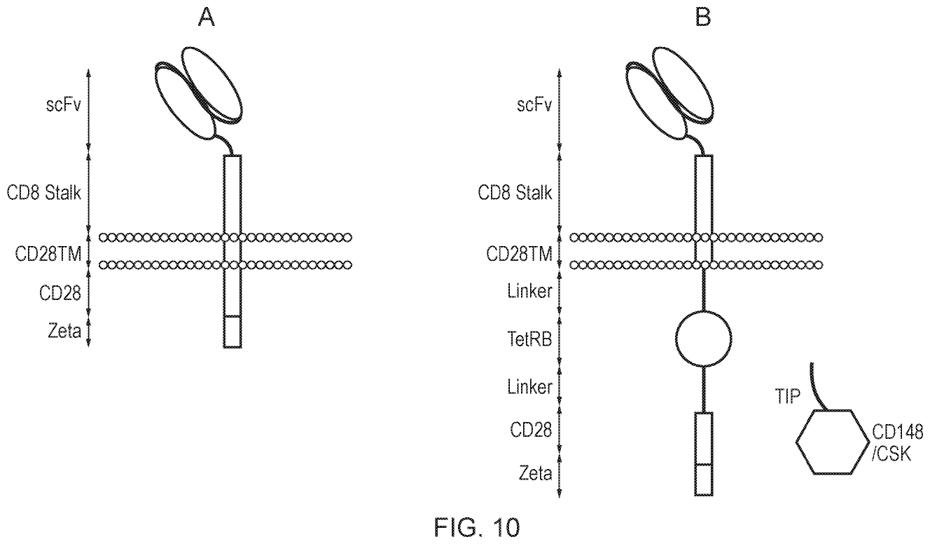

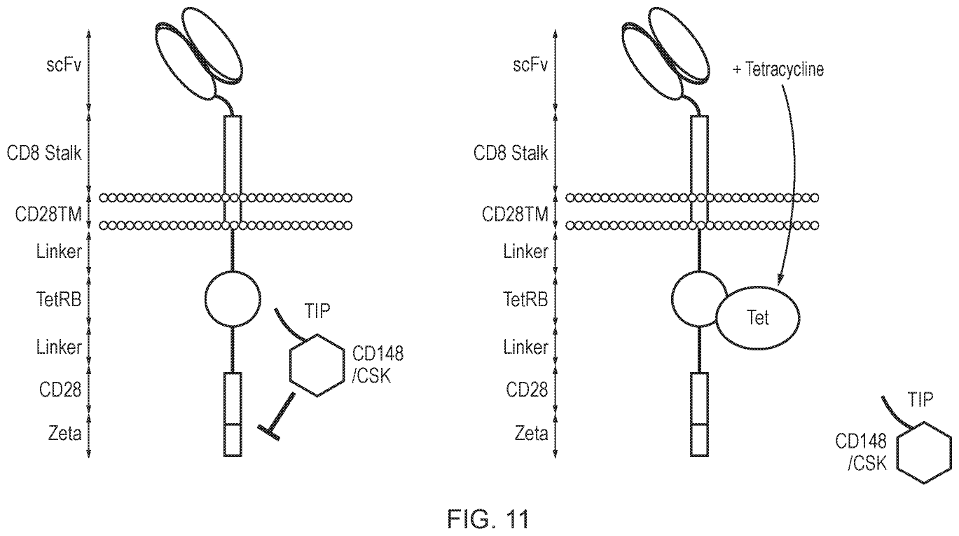

Additional aspects of the invention, relating to the “fused dimerising dampener” embodiment of the invention illustrated in FIGS. 8 to 11 are summarised in the following numbered paragraphs A1 to A34.

A1. A cell which comprises;

(i) a chimeric antigen receptor (CAR) comprising an antigen binding domain, a transmembrane domain, a first dimerization domain and an intracellular signalling domain; and

(ii) a signal-dampening component (SDC) comprising a signal-dampening domain (SDD) and a second dimerization domain which specifically binds the first dimerisation domain of the CAR.

A2. A cell according to paragraph A1, wherein the SDD inhibits the intracellular signalling domain of the CAR.

A3. A cell according to paragraph A2, wherein the SDD comprises a phosphatase domain capable of dephosphorylating immunoreceptor tyrosine-based activation motifs (ITAMs).

A4. A cell according to paragraph A3, wherein the SDD comprises the endodomain of CD148 or CD45.

A5. A cell according to paragraph A3, wherein the SDD comprises the phosphatase domain of SHP-1 or SHP-2

A6. A cell according to paragraph A2, wherein the SDD comprises an immunoreceptor tyrosine-based inhibition motif (ITIM).

A7. A cell according to paragraph A6, wherein the SDD comprises an endodomain from one of the following inhibitory receptors: PD1, BTLA, 2B4, CTLA-4, GP49B, Lair-1, Pir-B, PECAM-1, CD22, Siglec 7, Siglec 9, KLRG1, ILT2, CD94-NKG2A and CD5.

A8. A cell according to paragraph A2, wherein the SDD inhibits a Src protein kinase.

A9. A cell according to paragraph A8, wherein the SDD inhibits Lck.

A10. A cell according to paragraph A8 or A9, which comprises the kinase domain of CSK.

A11. A cell according to paragraph A1, wherein the SDD causes the removal of the intracellular signalling domain of the CAR.

A12. A cell according to paragraph A11, wherein the SDD comprises a protease and the CAR comprises a protease cleavage site.

A13. A cell according to paragraph A12, wherein the SDD comprises Tobacco Etch Virus Protease (TeV).

A14. A cell according to any preceding paragraph wherein binding of the first and second dimerization domains is controllable by the presence or absence of an agent.

A15. A cell according to paragraph A14, wherein binding of the first and second dimerization domains is induced by the presence of a chemical inducer of dimerisation (CID).

A16. A cell according to paragraph A15 wherein one dimerization domain comprises an FK506-binding protein (FKBP), the other dimerization domain comprises an FRB domain of mTOR and the CID is rapamycin or a rapamycin analogue.

A17. A cell according to paragraph A15, wherein the first and second dimerization domains comprise a FK506-binding protein (FKBP) and the CID is FK1012.

A18. A cell according to paragraph A15, wherein the first and second dimerization domains comprise GyrB and the CID is coumermycin or a derivative thereof.

A19. A cell according to paragraph A15 wherein one dimerization domain comprises GAI, the other dimerization domain comprises GID1 and the CID is gibberellin or a derivative thereof.

A20. A cell according to paragraph A14, wherein the agent disrupts binding of the first and second dimerization domains

A21. A cell according to paragraph A20, wherein one dimerization domain comprises the Tet repressor (TetR), the other dimerization domain comprises TetR interacting protein (TiP) and the agent is tetracycline, doxycycline, minocycline or an analogue thereof.

A22. A nucleic acid construct which comprises:
(i) a first nucleic acid sequence which encodes a chimeric antigen receptor (CAR) as defined in any preceding paragraph; and
(ii) a second nucleic acid sequence which encodes a signal-dampening component (SDC) as defined in any preceding paragraph.

A23. A nucleic acid construct according to paragraph 22, which has one of the following structures:

AgBD-TM-DD1-endo-coexpr-SDD-DD2;

AgBD-TM-endo-DD1-coexpr-SDD-DD2;

SDD-DD2-coexpr-AgBD-TM-DD1-endo;

SDD-DD2-coexpr-AgBD-TM-endo-DD1;

AgBD-TM-DD1-endo-coexpr-DD2-SDD;

AgBD-TM-endo-DD1-coexpr-DD2-SDD;

DD2-SDD-coexpr-AgBD-TM-DD1-endo; and

DD2-SDD-coexpr-AgBD-TM-endo-DD1

wherein

AgBD is a sequence encoding the antigen binding domain of the CAR

TM is a sequence encoding the transmembrane domain of the CAR

DD1 is a sequence encoding the first dimerization domain

Endo is a sequence encoding the intracellular signalling domain on the CAR

Coexpr is a sequence enabling the co-expression of the CAR and the SDC

SDD is a sequence encoding the signal-dampening domain of the SDC DD2 is a sequence encoding the second dimerization domain.

A24. A kit of nucleic acid sequences comprising:
(i) a first nucleic acid sequence which encodes a chimeric antigen receptor (CAR) as defined in any of paragraphs A1 to A21; and
(ii) a second nucleic acid sequence which encodes a signal-dampening component (SDC) as defined in any of paragraphs A1 to A21.

A25. A vector comprising a nucleic acid construct according to paragraph A22 or A23.

A26. A kit of vectors which comprises:
(i) a first vector which comprises a nucleic acid sequence which encodes a chimeric antigen receptor (CAR) as defined in any of paragraphs A1 to A21; and
(ii) a second vector which comprises a nucleic acid sequence which encodes a signal-dampening component (SDC) as defined in any of paragraphs A1 to A21.

A27. A pharmaceutical composition comprising a plurality of cells according to any of paragraphs 1 to 20.

A28. A pharmaceutical composition according to paragraph A27 for use in treating and/or preventing a disease.

A29, A method for treating and/or preventing a disease, which comprises the step of administering a pharmaceutical composition according to paragraph A27 to a subject.

A30. A method according to paragraph 28, which comprises the following steps:
(i) isolation of a cell-containing sample;
(ii) transduction or transfection of the cells with a nucleic acid construct according to paragraph A22 or A23, a kit of nucleic acid sequences according to paragraph A24; a vector according to paragraph A25 or a kit of vectors according to paragraph A26; and
(iii) administering the cells from (ii) to a subject.

A31, A method for controlling the activation of a cell according to any of paragraphs A1 to A21 in a subject, which comprises the step of administering an agent which controls binding or dissociation of the first and second dimerization domains to the subject.

A32. The use of a pharmaceutical composition according to paragraph A27 in the manufacture of a medicament for the treatment and/or prevention of a disease.

A33. The pharmaceutical composition for use according to paragraph A28, a method according to paragraph A29 or A30, of the use according to paragraph A32, wherein the disease is cancer.

A34. A method for making a cell according to any of paragraphs 1 to 20, which comprises the step of introducing a nucleic acid construct according to paragraph A22 or A23, a kit of nucleic acid sequences according to paragraph A24; a vector according to paragraph A25 or a kit of vectors according to paragraph A26 into a cell.

A35. A method according to paragraph A34 wherein the cell is from a sample isolated from a subject.

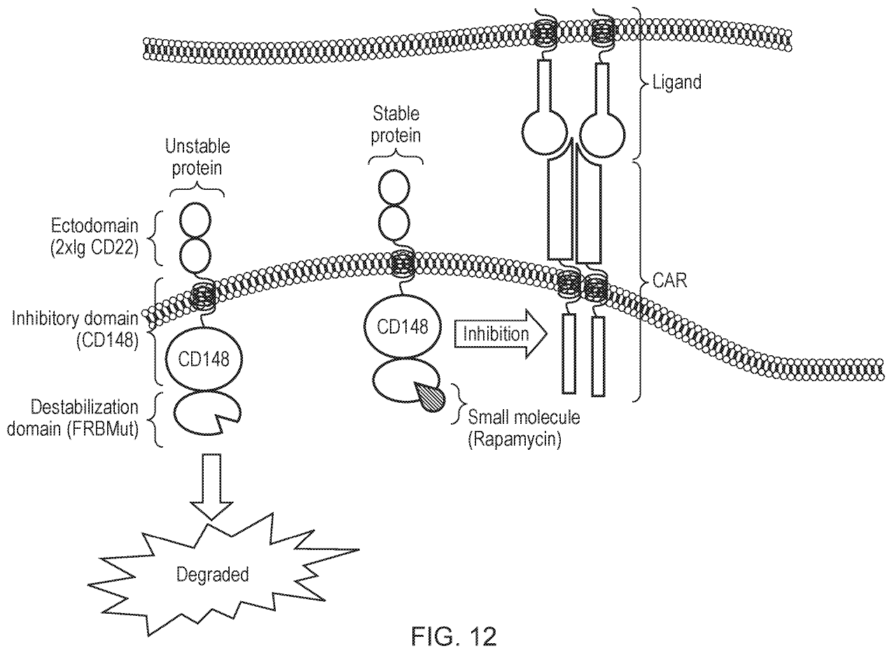

Additional aspects of the invention, relating to the “destabilisation domain dampener” embodiment of the invention illustrated in FIG. 12 are summarised in the following numbered paragraphs B1 to B29

B1. A cell which comprises;

(i) a chimeric antigen receptor (CAR) which comprises an antigen binding domain and an intracellular signalling domain; and

(iii) a membrane-tethered signal-dampening component (SDC) comprising a signal-dampening domain (SDD) and a destabilisation domain.

B2. A cell according to paragraph B1, wherein the SDD inhibits the intracellular signalling domain of the CAR.

B3. A cell according to paragraph B2, wherein the SDD comprises a phosphatase domain capable of dephosphorylating immunoreceptor tyrosine-based activation motifs (ITAMs).

B4. A cell according to paragraph B3, wherein the SDD comprises the endodomain of CD148 or CD45.

B5. A cell according to paragraph B3, wherein the SDD comprises the phosphatase domain of SHP-1 or SHP-2

B6. A cell according to paragraph B2, wherein the SOD comprises an immunoreceptor tyrosine-based inhibition motif (ITIM).

B7. A cell according to paragraph B6, wherein the SDD comprises an endodomain from one of the following inhibitory receptors: PD1, BTLA, 2B4, CTLA-4, GP49B, Lair-1, Pir-B, PECAM-1, CD22, Siglec 7, Siglec 9, KLRG1, ILT2, CD94-NKG2A and CD5.

B8. A cell according to paragraph B2, wherein the SDD inhibits a Src protein kinase.

B9. A cell according to paragraph B8, wherein the SDD inhibits Lck.

B10. A cell according to paragraph B8 or B9, which comprises the kinase domain of CSK.

B11. A cell according to paragraph 1, wherein the SDD causes the removal of the intracellular signalling domain of the CAR.

B12. A cell according to paragraph B11, wherein the SDD comprises a protease and the CAR comprises a protease cleavage site.

B13, A cell according to paragraph B12, wherein the SDD comprises Tobacco Etch Virus Protease (TeV).

B14. A cell according to any preceding paragraph wherein the destabilisation domain is stabilised by the presence of an agent, such that, in the presence of agent, the level of SDC is increased at the cell membrane.

B15. A cell according to paragraph B14 wherein the destabilisation domain comprises mutant FRB and the agent is rapamycin.

B16. A cell according to any preceding paragraph, wherein the membrane tethered SDC comprises a transmembrane domain or a myristoylation sequence,

B17. A nucleic acid construct which comprises:
(i) a first nucleic acid sequence which encodes a chimeric antigen receptor (CAR) as defined in any preceding paragraph; and
(ii) a second nucleic acid sequence which encodes a membrane-tethered signal-dampening component (SDC) as defined in any preceding paragraph.

B18. A kit of nucleic acid sequences comprising:
(i) a first nucleic acid sequence which encodes a chimeric antigen receptor (CAR) as defined in any of paragraphs B1 to B16;
(ii) a second nucleic acid sequence which encodes a membrane-tethered signal-dampening component (SDC) as defined in any of paragraphs B1 to B16.

B19. A vector comprising a nucleic acid construct according to paragraph B17,

B20. A kit of vectors which comprises:
(i) a first vector which comprises a nucleic acid sequence which encodes a chimeric antigen receptor (CAR) as defined in any of paragraphs B1 to B16;
(ii) a second vector which comprises a nucleic acid sequence which encodes a membrane-tethered signal-dampening component (SDC) as defined in any of paragraphs B1 to B16.

B21. A pharmaceutical composition comprising a plurality of cells according to any of paragraphs B1 to B16.

B22. A pharmaceutical composition according to paragraph B21 for use in treating and/or preventing a disease.

B23. A method for treating and/or preventing a disease, which comprises the step of administering a pharmaceutical composition according to paragraph B21 to a subject.

B24, A method according to paragraph B23, which comprises the following steps:
(i) isolation of a cell-containing sample;
(ii) transduction or transfection of the cells with a nucleic acid construct according to paragraph B17, a kit of nucleic acid sequences according to paragraph B18; a vector according to paragraph B19 or a kit of vectors according to paragraph B20; and
(iii) administering the cells from (ii) to a subject.

B25. A method for dampening CAR-mediated activation of a cell according to any of paragraphs B1 to B16 in a subject, which comprises the step of administering an agent which stabilises the destabilisation domain to the subject.

B26. A method according to paragraph B25, wherein the agent is rapamycin,

B27. The use of a pharmaceutical composition according to paragraph B21 in the manufacture of a medicament for the treatment and/or prevention of a disease.

B28. The pharmaceutical composition for use according to paragraph B22, a method according to paragraph B23 or B24, of the use according to paragraph B27, wherein the disease is cancer.

B29. A method for making a cell according to any of paragraphs B1 to B16, which comprises the step of introducing a nucleic acid construct according to paragraph B17, a kit of nucleic acid sequences according to paragraph B18; a vector according to paragraph B19 or a kit of vectors according to paragraph B20, into a cell.

B30. A method according to paragraph B29 wherein the cell is from a sample isolated from a subject.

### DETAILED DESCRIPTION

Chimeric Antigen Receptors (CAR)

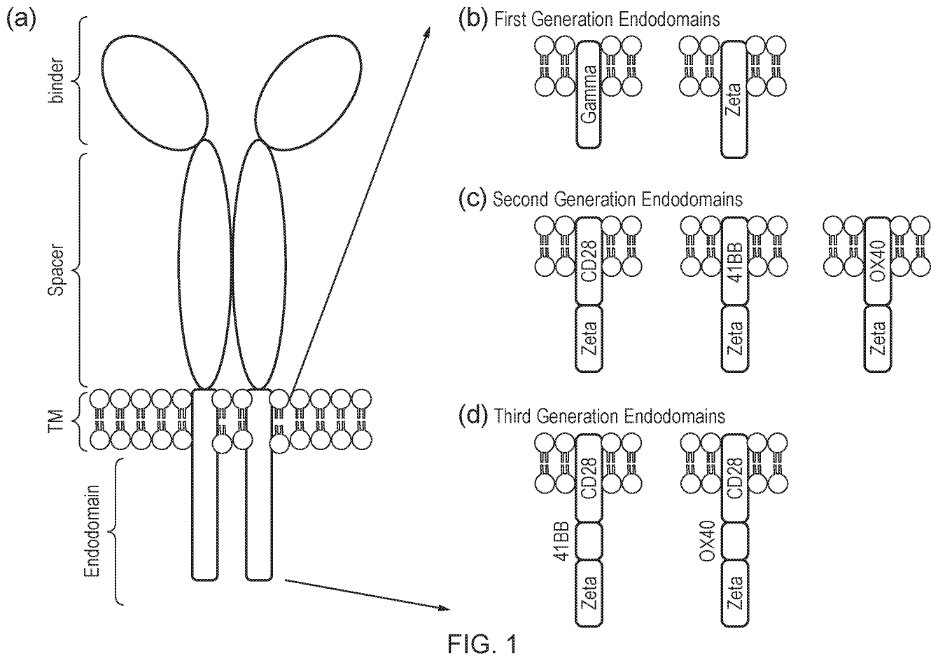

Classical CARs, which are shown schematically in FIG. 1, are chimeric type I trans-membrane proteins which connect an extracellular antigen-recognizing domain (binder) to an intracellular signalling domain (endodomain). The binder is typically a single-chain variable fragment (scFv) derived from a monoclonal antibody (mAb), but it can be based on other formats which comprise an antibody-like antigen binding site or on a ligand for the target antigen. A spacer domain may be necessary to isolate the binder from the membrane and to allow it a suitable orientation. A common spacer domain used is the Fc of IgG1. More compact spacers can suffice e.g. the stalk from CD8a and even just the IgG1 hinge alone, depending on the antigen. A trans-membrane domain anchors the protein in the cell membrane and connects the spacer to the endodomain.

Early CAR designs had endodomains derived from the intracellular parts of either the γ chain of the FcεR1 or CD3ζ. Consequently, these first generation receptors transmitted immunological signal 1, which was sufficient to trigger T-cell killing of cognate target cells but failed to fully activate the T-cell to proliferate and survive. To overcome this limitation, compound endodomains have been constructed: fusion of the intracellular part of a T-cell co-stimulatory molecule to that of CD3ζ results in second generation receptors which can transmit an activating and co-stimulatory signal simultaneously after antigen recognition. The co-stimulatory domain most commonly used is that of CD28. This supplies the most potent co-stimulatory signal namely immunological signal 2, which triggers T-cell proliferation. Some receptors have also been described which include TNF receptor family endodomains, such as the closely related OX40 and 41BB which transmit survival signals. Even more potent third generation CARs have now been described which have endodomains capable of transmitting activation, proliferation and survival signals.

CAR-encoding nucleic acids may be transferred to T cells using, for example, retroviral vectors. In this way, a large number of antigen-specific T cells can be generated for adoptive cell transfer. When the CAR binds the target-antigen, this results in the transmission of an activating signal to the T-cell it is expressed on. Thus the CAR directs the specificity and cytotoxicity of the T cell towards cells expressing the targeted antigen.

Antigen Binding Domain

The antigen-binding domain is the portion of a classical CAR which recognizes antigen.

Numerous antigen-binding domains are known in the art, including those based on the antigen binding site of an antibody, antibody mimetics, and T-cell receptors. For example, the antigen-binding domain may comprise: a single-chain variable fragment (scFv) derived from a monoclonal antibody; a natural ligand of the target antigen; a peptide with sufficient affinity for the target; a single domain binder such as a camelid; an artificial binder single as a Darpin; or a single-chain derived from a T-cell receptor.

Various tumour associated antigens (TAA) are known, as shown in the following Table 1. The antigen-binding domain used in the present invention may be a domain which is capable of binding a TAA as indicated therein.

TABLE 1

| Cancer type | TAA |
|---|---|
| Diffuse Large B-cell Lymphoma | CD19, CD20 |
| Breast cancer | ErbB2, MUC1 |
| AML | CD13, CD33 |
| Neuroblastoma | GD2, NCAM, ALK, GD2 |
| B-CLL | CD19, CD52, CD160 |
| Colorectal cancer | Folate binding protein, CA-125 |
| Chronic Lymphocytic Leukaemia | CD5, CD19 |
| Glioma | EGFR, Vimentin |
| Multiple myeloma | BCMA, CD138 |
| Renal Cell Carcinoma | Carbonic anhydrase IX, G250 |
| Prostate cancer | PSMA |
| Bowel cancer | A33 |

The antigen-binding domain may comprise a proliferation-inducing ligand (APRIL) which binds to B-cell membrane antigen (BCMA) and transmembrane activator and calcium modulator and cyclophilin ligand interactor (TACI). A CAR comprising an APRIL-based antigen-binding domain is described in WO2015/052538.

Transmemebrane Domain

The transmembrane domain is the sequence of a classical CAR that spans the membrane. It may comprise a hydrophobic alpha helix. The transmembrane domain may be derived from CD28, which gives good receptor stability.

Signal Peptide

The CAR may comprise a signal peptide so that when it is expressed in a cell, such as a T-cell, the nascent protein is directed to the endoplasmic reticulum and subsequently to the cell surface, where it is expressed.

The core of the signal peptide may contain a long stretch of hydrophobic amino acids that has a tendency to form a single alpha-helix. The signal peptide may begin with a short positively charged stretch of amino acids, which helps to enforce proper topology of the polypeptide during translocation. At the end of the signal peptide there is typically a stretch of amino acids that is recognized and cleaved by signal peptidase. Signal peptidase may cleave either during or after completion of translocation to generate a free signal peptide and a mature protein. The free signal peptides are then digested by specific proteases.

Spacer Domain

The CAR may comprise a spacer sequence to connect the antigen-binding domain with the transmembrane domain. A flexible spacer allows the antigen-binding domain to orient in different directions to facilitate binding.

The spacer sequence may, for example, comprise an IgG1 Fc region, an IgG1 hinge or a human CD8 stalk or the mouse CD8 stalk. The spacer may alternatively comprise an alternative linker sequence which has similar length and/or domain spacing properties as an IgG1 Fc region, an IgG1 hinge or a CD8 stalk. A human icy IgG1 spacer may be altered to remove Fc binding motifs.

Intracellular Signalling Domain

The intracellular signalling domain is the signal-transmission portion of a classical CAR.

The most commonly used signalling domain component is that of CD3-zeta endodomain, which contains 3 ITAMs. This transmits an activation signal to the T cell after antigen is bound. CD3-zeta may not provide a fully competent activation signal and additional co-stimulatory signalling may be needed. For example, chimeric CD28 and OX40 can be used with CD3-Zeta to transmit a proliferative/survival signal, or all three can be used together (illustrated in FIG. 1B).

The CAR may comprise the sequence shown as SEQ ID NO: 1, 2 or 3 or a variant thereof having at least 80% sequence identity.

SEQ ID NO: 1 - CD3 Z endodomain
RVKFSRSADAPAYQQGQNQLYNELNLGRREEYDVLDKRRGRDPEMGGKPRR
KNPQEGLYNELQKDKMAEAYSEIGMKGERRRGKGHDGLYQGLSTATKDTYD
ALHMQALPPR
SEQ ID NO: 2 - CD28 and CD3 Zeta endodomains
SKRSRLLHSDYMNMTPRRPGPTRKHYQPYAPPRDFAAYRSRVKFSRSADAP
AYQQGQNQLYNELNLGRREEYDVLDKRRGRDPEMGGKPRRKNPQEGLYNEL
QKDKMAEAYSEIGMKGERRRGKGHDGLYQGLSTATKDTYDALHMQALPPR
SEQ ID NO: 3 - CD28, OX40 and CD3 Zeta endodomains
SKRSRLLHSDYMNMTPRRPGPTRKHYQPYAPPRDFAAYRSRDQRLPPDAHK
PPGGGSFRTPIQEEQADAHSTLAKIRVKFSRSADAPAYQQGQNQLYNELNL
GRREEYDVLDKRRGRDPEMGGKPRRKNPQEGLYNELQKDKMAEAYSEIGMK
GERRRGKGHDGLYQGLSTATKDTYDALHMQALPPR

A variant sequence may have at least 80%, 85%, 90%, 95%, 98% or 99% sequence identity to SEQ ID NO: 1, 2 or 3, provided that the sequence provides an effective intracellular signalling domain.

Membrane Tethering Component (MTC)

The membrane tethering component acts as an anchor, tethering the first dimerization domain and therefore the signal dampening component to the intracellular surface of the cell membrane.

The membrane tethering component comprises a first heterodimerisation domain which interacts with a reciprocal domain on the signal dampening component.

The membrane tethering component may comprise a membrane localisation domain. This may be any sequence which causes the first dimerization domain to be attached to or held in a position proximal to the plasma membrane.

The membrane localisation domain may be or comprise a sequence which causes the nascent polypeptide to be attached initially to the ER membrane. As membrane material “flows” from the ER to the Golgi and finally to the plasma membrane, the protein remain associated with the membrane at the end of the synthesis/translocation process.

The membrane localisation domain may, for example, comprise a transmembrane sequence, a stop transfer sequence, a GPI anchor or a myristoylation/prenylation/palmitoylation site.

Alternatively the membrane localisation domain may direct the membrane-tethering component to a protein or other entity which is located at the cell membrane, for example by binding the membrane-proximal entity. The membrane tethering component may, for example, comprise a domain which binds a molecule which is involved in the immune synapse, such as TCR/CD3, CD4 or CD8.

Myristoylation is a lipidation modification where a myristoyl group, derived from myristic acid, is covalently attached by an amide bond to the alpha-amino group of an N-terminal glycine residue. Myristic acid is a 14-carbon saturated fatty acid also known as n-Tetradecanoic acid. The modification can be added either co-translationally or post-translationally. N-myristoyltransferase (NMT) catalyzes the myristic acid addition reaction in the cytoplasm of cells. Myristoylation causes membrane targeting of the protein to which it is attached, as the hydrophobic myristoyl group interacts with the phospholipids in the cell membrane.

The membrane tethering component of the present invention may comprise a sequence capable of being myristoylated by a NMT enzyme. The membrane tethering component of cell of the present invention may comprise a myristoyl group when expressed in a cell.

The membrane tethering component may comprise a consensus sequence such as: NH2-G1-X2-X3-X4-S5-X6-X7-X8 which is recognised by NMT enzymes.

Palmitoylation is the covalent attachment of fatty acids, such as palmitic acid, to cysteine and less frequently to serine and threonine residues of proteins. Palmitoylation enhances the hydrophobicity of proteins and can be used to induce membrane association. In contrast to prenylation and myristoylation, palmitoylation is usually reversible (because the bond between palmitic acid and protein is often a thioester bond). The reverse reaction is catalysed by palmitoyl protein thioesterases.

In signal transduction via G protein, palmitoylation of the α subunit, prenylation of the γ subunit, and myristoylation is involved in tethering the G protein to the inner surface of the plasma membrane so that the G protein can interact with its receptor.

The membrane tethering component may comprise a sequence capable of being palmitoylated. The membrane tethering component may comprise additional fatty acids when expressed in a cell which causes membrane localisation.

Prenylation (also known as isoprenylation or lipidation) is the addition of hydrophobic molecules to a protein or chemical compound. Prenyl groups (3-methyl-but-2-en-1-yl) facilitate attachment to cell membranes, similar to lipid anchors like the GPI anchor.

Protein prenylation involves the transfer of either a farnesyl or a geranyl-geranyl moiety to C-terminal cysteine(s) of the target protein. There are three enzymes that carry out prenylation in the cell, farnesyl transferase, Caax protease and geranylgeranyl transferase I.

The membrane tethering component may comprise a sequence capable of being prenylated. The membrane-tethering component may comprise one or more prenyl groups when expressed in a cell which causes membrane localisation.

Signal Dampening Component

The signal-dampening component (SDC) of the cell of the present invention comprises a signal-dampening domain (SDD) and a second dimerization domain. The second dimerization domain specifically binds the first dimerisation domain of the membrane-tethering component.

The signal-dampening domain inhibits CAR-mediated cell signalling when located on the intracellular side of the cell membrane and therefore located proximal to the CAR endodomain.

The signal dampening domain may inhibit CAR-mediated cell signalling completely, effectively “turning off” CAR mediated cell activation. Alternatively the SDD may cause partial inhibition, effectively “turning down” CAR-mediated cell signalling.

The presence of the signal dampening domain may result in signalling through the signalling component which is 2, 5, 10, 50, 100, 1,000 or 10,000-fold lower than the signalling which occurs in the absence of the signal dampening domain.

CAR mediated signalling may be determined by a variety of methods known in the art. Such methods include assaying signal transduction, for example assaying levels of specific protein tyrosine kinases (PTKs), breakdown of phosphatidylinositol 4,5-biphosphate (PIP2), activation of protein kinase C (PKC) and elevation of intracellular calcium ion concentration. Functional readouts, such as clonal expansion of T cells, upregulation of activation markers on the cell surface, differentiation into effector cells and induction of cytotoxicity or cytokine (e.g. IL-2) secretion may also be utilised.

Control of T Cell Signalling

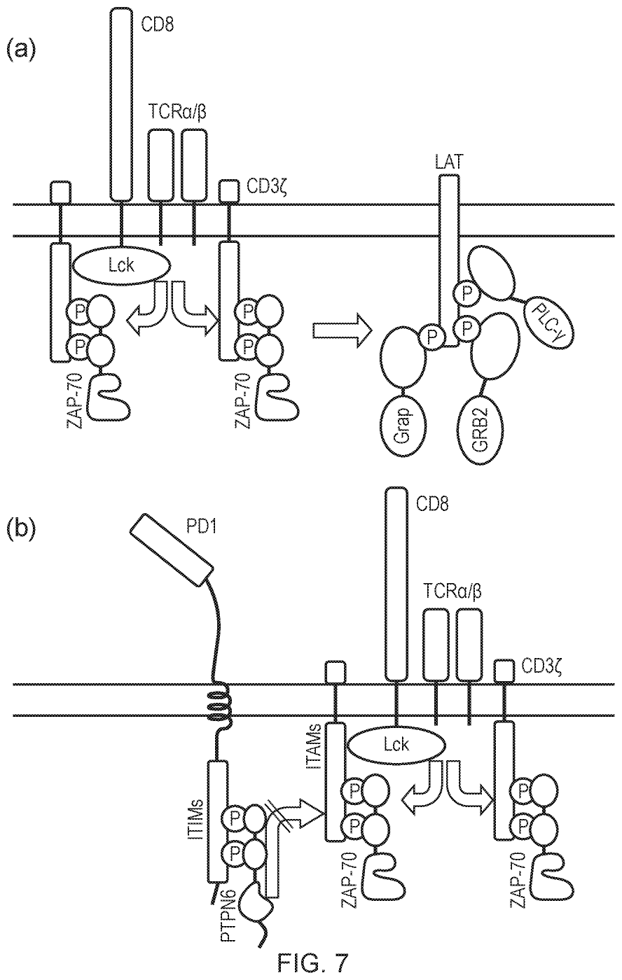

The earliest step in T cell activation is the recognition of a peptide MHC-complex on the target cell by the TCR. This initial event causes the close association of Lck kinase with the cytoplasmic tail of CD3-zeta in the TCR complex. Lck then phosphorylates immunoreceptor tyrosine-based activation motifs (ITAMs) in the cytoplasmic tail of CD3-zeta which allows the recruitment of ZAP70. ZAP70 is an SH2 containing kinase that plays a pivotal role in T cell activation following engagement of the TCR. Tandem SH2 domains in ZAP70 bind to the phosphorylated CD3 resulting in ZAP70 being phosphorylated and activated by Lck or by other ZAP70 molecules in trans. Active ZAP70 is then able to phosphorylate downstream membrane proteins, key among them the linker of activated T cells (LAT) protein. LAT is a scaffold protein and its phosphorylation on multiple residues allows it to interact with several other SH2 domain-containing proteins including Grb2, PLC-g and Grap which recognize the phosphorylated peptides in LAT and transmit the T cell activation signal downstream ultimately resulting in a range of T cell responses. This process is summarized in FIG. 7A.

T cell activation is controlled by kinetic segregation or molecules at the T-cell:target cell synapse. At the ground state, the signalling components on the T-cell membrane are in dynamic homeostasis whereby dephosphorylated ITAMs are favoured over phosphorylated ITAMs. This is due to greater activity of the transmembrane CD45/CD148 phosphatases over membrane-tethered kinases such as lck. When a T-cell engages a target cell through a T-cell receptor (or CAR) recognition of cognate antigen, tight immunological synapses form. This close juxtapositioning of the T-cell and target membranes excludes CD45/CD148 due to their large ectodomains which cannot fit into the synapse. Segregation of a high concentration of T-cell receptor associated ITAMs and kinases in the synapse, in the absence of phosphatases, leads to a state whereby phosphorylated ITAMs are favoured. ZAP70 recognizes a threshold of phosphorylated ITAMs and propagates a T-cell activation signal.

In vivo, membrane-bound immunoinhibitory receptors such as CTLA4, PD-1, LAG-3, 2B4 or BTLA 1 also inhibit T cell activation. As illustrated schematically in FIG. 7B, inhibitory immune-receptors such as PD1 effectively reverse the first steps of the T-cell activation process. PD1 has ITIMs in its endodomain which are recognized by the SH2 domains of SHP-1 or SHP-2. Upon recognition, SHP-1 and/or SHP-2 is recruited to the juxta-membrane region and its phosphatase domain subsequently de-phosphorylates ITAM domains inhibiting immune activation.

Phosphatases

The signal dampening domain of the signal dampening component may comprise a phosphatase, such as a phosphatase capable of dephosphorylating an ITAM.

The signal dampening domain of the signal dampening component may comprise all of part of a receptor-like tyrosine phosphatase. The phospatase may interfere with the phosphorylation and/or function of elements involved in T-cell signalling, such as PLCγ1 and/or LAT.

The signal dampening domain may comprise the phosphatase domain of one or more phosphatases which are involved in controlling T-cell activation, such as CD148, CD45, SHP-1 or SHP-2.

CD148

CD148 is a receptor-like protein tyrosine phosphatase which negatively regulates TCR signaling by interfering with the phosphorylation and function of PLCy1 and LAT.

The endodomain of CD148 is shown as SEQ ID No. 4,

SEQ ID No 4 - CD148 endodomain sequence
RKKRKDAKNNEVSFSQIKPKKSKLIRVENFEAYFKKQQADSNCGFAEEYED
LKLVGISQPKYAAELAENRGKNRYNNVLPYDISRVKLSVQTHSTDDYINAN
YMPGYHSKKDFIATQGPLPNTLKDFWRMVWEKNVYAIIMLTKCVEQGRTKC
EEYWPSKQAQDYGDITVAMTSEIVLPEWTIRDFTVKNIQTSESHPLRQFHF
TSWPDHGVPDTTDLLINFRYLVRDYMKQSPPESPILVHCSAGVGRTGTFIA
IDRLIYQIENENTVDVYGIVYDLRMHRPLMVQTEDQYVFLNQCVLDIVRSQ
KDSKVDLIYQNTTAMTIYENLAPVTTFGKTNGYIA

CD45

CD45 present on all hematopoetic cells, is a protein tyrosine phosphatase which is capable of regulating signal transduction and functional responses, again by phosphorylating PLC γ1.

The endodomain of CD45 is shown as SEQ ID No, 5.

SEQ ID 5 - CD45 endodomain sequence
KIYDLHKKRSCNLDEQQELVERDDEKQLMNVEPIHADILLETYKRKIADEG
QSRLFLAEFIPRVFSKFPIKEARKPFNQNKNRYVDILPYDYNRVELSEING
DAGSNYINASYIDGFKEPRKYIAAQGPRDETVDDFWRMIWEQKATVIVMVT
RCEEGNRNKCAEYVVPSMEEGTRAFGDVVVKINQHKRCPDYIIQKLNIVNK
KEKATGREVTHIQFTSWPDHGVPEDPHLLLKLRRRVNAFSNFFSGPIVVHC
SAGVGRTGTYIGIDAMLEGLEAENKVDVYGYVVKLRRQRCLMVQVEAQYIL
IHQALVEYNQFGETEVNLSELHPYLHNMKKRDPPSEPSPLEAEFQRLPSYR
SWRTQHIGNQEENKSKNRNSNVIPYDYNRVPLKHELEMSKESEHDSDESSD
DDSDSEEPSKYINASFIMSYWKPEVMIAAQGPLKETIGDFWQMIFQRKVKV
IVMLTELKHGDQEICAQYWGEGKQTYGDIEVDLKDTDKSSTYTLRVFELRH
SKRKDSRTVYQYQYTNWSVEQLPAEPKELISMIQVVKQKLPQKNSSEGNKH
HKSTPLLIHCRDGSQQTGIFCALLNLLESAETEEVVDIFQVVKALRKARPG
MVSTFEQYQFLYDVIASTYPAQNGQVKKNNHQEDKIEFDNEVDKVKQDANC
VNPLGAPEKLPEAKEQAEGSEPTSGTEGPEHSVNGPASPALNQGS

SHP1/SHP2

Src homology region 2 domain-containing phosphatase-1 (SHP-1, also known as PTPN6) is a member of the protein tyrosine phosphatase family.

The N-terminal region of SHP-1 contains two tandem SH2 domains which mediate the interaction of PTPN6 and its substrates. The C-terminal region contains a tyrosine-protein phosphatase domain.

SHP-1 is capable of binding to, and propagating signals from, a number of inhibitory immune receptors or ITIM containing receptors, such as, PD1, PDCD1, BTLA4, LILRB1, LAIR1, CTLA4, KIR2DL1, KIR2DL4, KIR2DL5, KIR3DL1 and KIR3DL3.

Human SHP-1 protein has the UniProtKB accession number P29350.

The protein tyrosine phosphatase (PTP) domain of SHP-1 is shown below as sequence ID No. 6.

SHP-1 phosphatase domain
(SEQ ID NO: 6)
FWEEFESLQKQEVKNLHORLEGQRPENKGKNRYKNILPFDHSRVILQGRDS
NIPGSDYINANYIKNOLLGPDENAKTYIASQGCLEATVNDFWQMAWQENSR
VIVMTTREVEKGRNKCVPYWPEVGMQRAYGPYSVTNCGEHDTTEYKLRTLQ
VSPLDNGDLIREIWHYQYLSWPDHGVPSEPGGVLSFLDQINQRQESLPHAG
PIIVHCSAGIGRTGTIIVIDMLMENISTKGLDCDIDIQKTIQMVRAQRSGM
VQTEAQYKFIYVAIAQFIETTKKKLEVLQSQKGQESEYGNITYPRAMKNAH
AKASRTSSKHKEDWENLHTKNKREEKVKKQRSADKEKSKGSLKRK

SHP-2

SHP-2, also known as PTPN11, PTP-1D and PTP-2C is a member of the protein tyrosine phosphatase (PTP) family. Like PTPN6, SHP-2 has a domain structure that consists of two tandem SH2 domains in its N-terminus followed by a protein tyrosine phosphatase (PTP) domain. In the inactive state, the N-terminal SH2 domain binds the PTP domain and blocks access of potential substrates to the active site. Thus, SHP-2 is auto-inhibited. Upon binding to target phospho-tyrosyl residues, the N-terminal SH2 domain is released from the PTP domain, catalytically activating the enzyme by relieving the auto-inhibition.

Human SHP-2 has the UniProtKB accession number P35235-1.

The protein tyrosine phosphatase (PTP) domain of SHP-2 is shown below as sequence ID No, 7.

SHP-2 phosphatase domain
(SEQ ID NO: 7)
FWEEFETLQQQECKLLYSRKEGQRQENKNKNRYKNILPFDHTRVVLHDGDP
NEPVSDYINANIIMPEFETKCNNSKPKKSYIATQGCLQNTVNDFWRMVFQE
NSRVIVMTTKEVERGKSKCVKYWPDEYALKEYGVMRVRNVKESAAHDYTLR
ELKLSKVGQALLQGNTERTVWQYHFRTWPDHGVPSDPGGVLDFLEEVHHKQ
ESIVDAGPVVVHCSAGIGRTGTFIVIDILIDIIREKGVDCDIDVPKTIQMV
RSQRSGMVQTEAQYRFIYMAVQHYIETLQRRIEEEQKSKRKGHEYTNIKYS
LVDQTSGDQSPLPPCTPTPPCAEMREDSARVYENVGLMQQQRSFR

The signal dampening domain may comprise the phosphatase domain of SEQ ID No 4, 5, 6 or 7 or a variant thereof. The variant may, for example, have at least 80, 85, 90, 95, 98 or 99% sequence identity, provided that the variant sequence is capable of dampening CAR-mediated cell signalling. The variant phosphatase may be capable of dephosphorylating one or more ITAM(s).

Endodomains from Immunoregulatory Molecules

The signal dampening domain of the signal dampening component may comprise all or part of the endodomain of an immunoregulatory molecule which inhibits T cell signalling. For example, the signal dampening domain may comprise the endodomain from an immunoinhibitory receptor which inhibits T cell activation. The inhibitory receptor may be a member of the CD28 or Siglec family such as CTLA4, PD-1, LAG-3, 2B4, BTLA 1, CD28, ICOS. CD33, CD31, CD27, CD30, GITR or HVEM or Siglec-5, 6, 7, 8, 9, 10 or 11.

The signal dampening domain may comprise one or more immunoreceptor tyrosine-based inhibition motifs (ITIMs).

An ITIM is a conserved sequence of amino acids (S/IN/LxYxxI/V/L) that is found in the cytoplasmic tails of many inhibitory receptors of the immune system. After ITIM-possessing inhibitory receptors interact with their ligand, their ITIM motif becomes phosphorylated by enzymes of the Src kinases.

Immune inhibitory receptors such as PD1, PDCD1, BTLA4, LILRB1, LAIR1, CTLA4, 2B4, GP49B, Pir-B, PECAM-1, CD22, Siglec 7, Siglec 9, KLRG1, ILT2, CD94-NKG2A, CD5 and the Killer inhibitory receptor family (KIR) including KIR2DL1, KIR2DL4, KIR2DL5, KIR3DL1 and KIR3DL3 contain ITIMs.

The signal dampening domain may comprise one or more of the sequence(s) shown as SEQ ID NO: 8 to 24.

SEQ ID NO: 8 - ICOS endodomain
CWLTKKKYSSSVHDPNGEYMFMRAVNTAKKSRLTDVTL
SEQ ID NO: 9 - CD27 endodomain
QRRKYRSNKGESPVEPAEPCHYSCPREEEGSTIPIQEDYRKPEPACSP
SEQ ID NO: 10 - BTLA endodomain
RRHQGKQNELSDTAGREINLVDAHLKSEQTEASTRQNSQVLLSETGIYDND
PDLCFRMQEGSEVYSNPCLEENKPGIVYASLNHSVIGPNSRLARNVKEAPT
EYASICVRS
SEQ ID NO: 11 - CD30 endodomain
HRRACRKRIRQKLHLCYPVQTSQPKLELVDSRPRRSSTQLRSGASVTEPVA
EERGLMSQPLMETCHSVGAAYLESLPLQDASPAGGPSSPRDLPEPRVSTEH
TNNKIEKIYIMKADTVIVGTVKAELPEGRGLAGPAEPELEEELEADHTPHY
PEQETEPPLGSCSDVMLSVEEEGKEDPLPTAASGK
SEQ ID NO: 12 - GITR endodomain
QLGLHIWQLRSQCMWPRETQLLLEVPPSTEDARSCQFPEEERGERSAEEKG
RLGDLWV
SEQ ID NO: 13 - HVEM endodomain
CVKRRKPRGDVVKVIVSVQRKRQEAEGEATVIEALQAPPDVTTVAVEETIP
SFTGRSPNH
SEQ ID No. 14 - PD1 endodomain
CSRAARGTIGARRTGQPLKEDPSAVPVFSVDYGELDFQWREKTPEPPVPCV
PEQTEYATIVFPSGMGTSSPARRGSADGPRSAQPLRPEDGHCSWPL
SEQ ID No. 15 - PDCD1 endodomain
CSRAARGTIGARRTGQPLKEDPSAVPVFSVDYGELDFQWREKTPEPPVPCV
PEQTEYATIVFPSGMGTSSPARRGSADGPRSAQPLRPEDGHCSWPL
SEQ ID No. 16 - BTLA4 endodomain
KLQRRWKRTQSQQGLQENSSGQSFFVRNKKVRRAPLSEGPHSLGCYNPMME
DGISYTTLRFPEMNIPRIGDAESSEMQRPPPDCDDTVTYSALHKRQVGDYE
NVIPDFPEDEGIHYSELIQFGVGERPQAQENVDYVILKH
SEQ ID No. 17 - LILRB1 endodomain
LRHRRQGKHWTSTQRKADFQHPAGAVGPEPTDRGLQWRSSPAADAQEENLY
AAVKHTQPEDGVEMDTRSPHDEDPQAVTYAEVKHSRPRREMASPPSPLSGE
FLDTKDRQAEEDRQMDTEAAASEAPQDVTYAQLHSLTLRREATEPPPSQEG
PSPAVPSIYATLAIH
SEQ ID No. 18 - LAIR1 endodomain
HRQNQIKQGPPRSKDEEQKPQQRPDLAVDVLERTADKATVNGLPEKDRETD
TSALAAGSSQEVTYAQLDHWALTQRTARAVSPQSTKPMAESITYAAVARH
SEQ ID No. 19 - CTLA4 endodomain
FLLWILAAVSSGLFFYSFLLTAVSLSKMLKKRSPLTTGVYVKMPPTEPECE
KQFQPYFIPIN
SEQ ID No. 20 - KIR2DL1 endodomain
GNSRHLHVLIGTSVVIIPFAILLFFLLHRWCANKKNAVVMDQEPAGNRTVN
REDSDEQDPQEVTYTQLNHCVFTQRKITRPSQRPKTPPTDIIVYTELPNAE
SRSKVVSCP
SEQ ID No. 21 - KIR2DL4 endodomain
GIARHLHAVIRYSVAIILFTILPFFLLHRWCSKKKENAAVMNQEPAGHRTV
NREDSDEQDPQEVTYAQLDHCIFTQRKITGPSQRSKRPSTDTSVCIELPNA
EPRALSPAHEHHSQALMGSSRETTALSQTQLASSNVPAAGI
SEQ ID No. 22 - KIR2DL5 endodomain
TGIRRHLHILIGTSVAIILFIILFFFLLHCCCSNKKNAAVMDQEPAGDRTV
NREDSDDQDPQEVTYAQLDHCVFTQTKITSPSQRPKTPPTDTTMYMELPNA
KPRSLSPAHKHHSQALRGSSRETTALSQNRVASSHVPAAGI
SEQ ID No. 23 - KIR3DL1 endodomain
KDPRHLHILIGTSVVIILFILLLFFLLHLWCSNKKNAAVMDQEPAGNRTAN
SEDSDEQDPEEVTYAQLDHCVFTQRKITRPSQRPKTPPTDTILYTELPNAK
PRSKVVSCP
SEQ ID No. 24 - KIR3DL3 endodomain
KDPGNSRHLHVLIGTSVVIIPFAILLFFLLHRWCANKKNAVVMDQEPAGNR
TVNREDSDEQDPQEVTYAQLNHCVFTQRKITRPSQRPKTPPTDTSV

The signal dampening domain may comprise a variant of one of the sequences shown as SEQ ID NO: 8 to 24 having at least 80%, 85%, 90%, 95%, 98% or 99% sequence identity. The variant sequence may be able to recruit SHP-1 and/or SHP-2 to the cell membrane. The variant sequence may comprise one or more ITIM(s).

CSK Endodomain

Protein tyrosine kinases (PTKs) are signaling molecules that regulate a variety of cellular processes including cell growth, differentiation, mitotic cycle, and oncogenic transformation. The N-terminal part of non-receptor (or cytoplasmic) PTK contains two tandem Src homolog (SH2) domains, which act as protein phospho-tyrosine binding domains, and mediate the interaction of this PTK with its substrates. Tyrosine proteins kinases are a subclass of protein kinase, where the phosphate group is attached to the amino acid tyrosine on the protein.

Tyrosine-protein kinase CSK (C-terminal Src kinase) is an enzyme (UniProt ID: P41240 [http://www.uniprot.org/uniprot/P41240]) which phosphorylates tyrosine residues located in the C-terminal end of Src-family kinases (SFKs), such as SRC, HCK, FYN, LYN and notably LCK. CSK is mainly expressed in the lungs and macrophages as well as several other tissues. Tyrosine-kinase CSK is mainly present in the cytoplasm, but also found in lipid rafts making cell-cell junction.

CSK is a non-receptor tyrosine-protein kinase with molecular mass of 50 kDa. CSK plays an important role in the regulation of cell growth, differentiation, migration and immune response. CSK acts by suppressing the activity of the SFKs by phosphorylation of family members at a conserved C-terminal tail site.

CSK contains the SH3 and SH2 domains in its N-terminus and a kinase domain in its C-terminus. This arrangement of functional domains within the primary structure is similar to that of SFKs, but CSK lacks the N-terminal fatty acylation sites, the auto-phosphorylation site in the activation loop, and the C-terminal negative regulatory sites, all of which are conserved among SFK proteins and critical for their proper regulation. The absence of auto-phosphorylation in the activation loop is a distinguishing feature of CSK. The most striking feature of the CSK structure is that, unlike the situation in SFKs, the binding pockets of the SH3 and SH2 domains are oriented outward, enabling intermolecular interactions with other molecules. In active molecules, the SH2-kinase and SH2-SH3 linkers are tightly bound to the N-terminal lobe of the kinase domain in order to stabilize the active conformation, and there is a direct linkage between the SH2 and the kinase domains. In inactive molecules, the SH2 domains are rotated in a manner that disrupts the linkage to the kinase domain.

Upon phosphorylation by other kinases, Src-family members engage in intramolecular interactions between the phosphotyrosine tail and the SH2 domain that result in an inactive conformation. To inhibit SFKs, CSK is recruited to the plasma membrane via binding to transmembrane proteins or adapter proteins located near the plasma membrane and ultimately suppresses signaling through various surface receptors, including T-cell receptor (TCR) by phosphorylating and maintaining inactive several effector molecules.

Because Csk lacks a transmembrane domain and fatty acyl modifications, it is predominantly present in cytosol, whereas its substrate SFKs are anchored to the membrane via their N-terminal myristate and palmitate moieties. Therefore, the translocation of CSK to the membrane, where SFKs are activated, is thought to be a critical step of CSK regulation. So far, several scaffolding proteins, e.g., caveolin-1, paxillin, Dab2, VE-cadherin, IGF-1R, IR, LIME, and SIT1, have been identified as membrane anchors of CSK, as well intrinsic phosphoprotein Cbp/PAG1 (Csk binding protein/phosphoprotein associated with glycosphingolipid-enriched membrane). Cbp has a single transmembrane domain at its N-terminus and two palmitoyl modification sites just C-terminal to the transmembrane domain, through which Cbp is exclusively localized to lipid rafts.

A CSK endodomain may comprise all of CSK (SEQ ID No. 25) or just the tyrosine kinase domain (SEQ ID No. 26).

SEQ ID No: 25 - sequence of full length CSK
SAIQAAWPSGTECIAKYNFHGTAEQDLPFCKGDVLTIVAVTKDPNWYKAKN
KVGREGIIPANYVQKREGVKAGTKLSLMPWFHGKITREQAERLLYPPETGL
FLVRESTNYPGDYTLCVSCDGKVEHYRIMYHASKLSIDEEVYFENLMQLVE
HYTSDADGLCTRLIKPKVMEGTVAAQDEFYRSGWALNMKELKLLQTIGKGE
FGDVMLGDYRGNKVAVKCIKNDATAQAFLAEASVMTQLRHSNLVQLLGVIV
EEKGGLYIVTEYMAKGSLVDYLRSRGRSVLGGDCLLKFSLDVCEAMEYLEG
NNFVHRDLAARNVLVSEDNVAKVSDFGLTKEASSTQDTGKLPVKWTAPEAL
REKKFSTKSDVWSFGILLWEIYSFGRVPYPRIPLKDVVPRVEKGYKMDAPD
GCPPAVYEVMKNCWHLDAAMRPSFLQLREQLEHIKTHELHL
SEQ ID No: 26 - sequence of tyrosine kinase domain
of CSK
LKLLQTIGKGEFGDVMLGDYRGNKVAVKCIKNDATAQAFLAEASVMTQLRH
SNLVQLLGVIVEEKGGLYIVTEYMAKGSLVDYLRSRGRSVLGGDCLLKFSL
DVCEAMEYLEGNNFVHRDLAARNVLVSEDNVAKVSDFGLTKEASSTQDTGK
LPVKWTAPEALREKKFSTKSDVWSFGILLWEIYSFGRVPYPRIPLKDVVPR
VEKGYKMDAPDGCPPAVYEVMKNCWHLDAAMRPSFLQLREQLEHIKTHELH
L

A CSK endodomain may comprise a variant of the sequence shown as SEQ ID No. 25 or 26 or part thereof having at least 80% sequence identity, as long as the variant retains the capacity to inhibit T cell signaling by a CAR when brought into the vicinity of the CAR.

Removal of Intracellular Signalling Domain

The signal dampening domain may abrogate, reduce or block CAR-mediated CAR signalling by causing complete or partial removal of the intracellular signalling domain of the CAR.

For example, the SDD may comprises a protease and the CAR may comprise a protease cleavage site, for example between the transmembrane domain and the intracellular signalling domain; or within the intracellular signalling domain, such that cleavage reduces or removes the cell signalling capacity of the intracellular signalling domain.

Protease Domain

The protease domain may, for example, be any protease which is capable of cleaving at a specific recognition sequence. As such the protease domain may be any protease which enables the separation of a single target polypeptide into two distinct polypeptides via cleavage at a specific target sequence.

The protease domain may be a Tobacco Etch Virus (TeV) protease domain.

TeV protease is a highly sequence-specific cysteine protease which is chymotrypsin-like proteases. It is very specific for its target cleavage site and is therefore frequently used for the controlled cleavage of fusion proteins both in vitro and in vivo. The consensus TeV cleavage site is ENLYFQ\S (where ‘\’ denotes the cleaved peptide bond). Mammalian cells, such as human cells, do not express endogenous TeV protease.

The TeV cleavage recognition site is shown as SEQ ID NO: 27.

SEQ ID NO: 27 - Tev cleavage site
ENLYFQS

The TeV protease domain is shown as SEQ ID NO: 28.

SEQ ID NO: 28
SLFKGPRDYNPISSTICHLTNESDGHTTSLYGIGFGPFIITNKHLFRRNNG
TLLNQSLHGVFKVKNTTTLQQHLIDGRDMIIIRMPKDFPPFPQKLKFREPQ
REERICLVTTNFQTKSMSSMVSDTSCTFPSSDGIFWKHWIQTKDGQCGSPL
VSTRDGFIVGIHSASNFTNTNNYFTSVPKNFMELLTNQEAQQWVSGWRLNA
DSVLWGGHKVFMSKPEEPFQPVKEATQLMNELVYSQ

The protease domain may be or comprise the sequence shown as SEQ ID NO: 28, or a variant thereof having at least 80, 85, 90, 95, 98 or 99% sequence identity provided that the sequence provides an effective protease function.

Dimerisation Domains

In the cell of the present invention, the membrane tethering component comprises a first dimerization domain and the signal dampening component comprises a second dimerization domain and the first and second dimerization domains are capable of specific association.

The first and second dimerization domains may be any combination of domains which interact resulting in co-localization of the membrane tethering component and the signal dampening component at the cell membrane.

The first and second dimerization domains may be capable of spontaneous dimerization with each other. In this embodiment, dimerization occurs with the first and second heterodimerization domains alone, without the need for any separate molecule acting as an “inducer” of dimerization.

Various dimerization domains capable of spontaneous dimerization are known in the art, including leucine zippers; dimerization and docking domain (DDD1) and anchoring domain (AD1); Bacterial Ribonuclease (Barnase) and Barnstar peptides; and Human Pancreatic RNases and S-peptide. Further detail on these dimerization systems may be found in WO2016/124930.

Agent-Mediated Dimerisation

In a second embodiment, the first and second dimerization domains are capable of dimerising only in the presence of an agent i.e. a separate molecule acting as an “inducer” of dimerization.

The macrolides rapamycin and FK506 act by inducing the heterodimerization of cellular proteins. Each drug binds with a high affinity to the FKBP12 protein, creating a drug-protein complex that subsequently binds and inactivates mTOR/FRAP and calcineurin, respectively. The FKBP-rapamycin binding (FRB) domain of mTOR has been defined and applied as an isolated 89 amino acid protein moiety that can be fused to a protein of interest. Rapamycin can then induce the approximation of FRB fusions to FKBP12 or proteins fused with FKBP 12.

In the context of the present invention, one of the dimerization domains may comprise FRB or a variant thereof and the other dimerization domain may comprise FKBP12 or a variant thereof.

The dimerization domains may be or comprise one the sequences shown as SEQ ID NO: 29 to SEQ ID NO: 33.

SEQ ID No 29 - FKBP12 domain
MGVQVETISPGDGRTFPKRGQTCVVHYTGMLEDGKKFDSSRDRNKPFKFML
GKQEVIRGWEEGVAQMSVGQRAKLTISPDYAYGATGHPGIIPPHATLVFDV
ELLKLE
SEQ ID No 30 - wild-type FRB segment of mTOR
MASRILWHEMWHEGLEEASRLYFGERNVKGMFEVLEPLHAMMERGPQTLKE
TSFNQAYGRDLMEAQEWCRKYMKSGNVKDLTQAWDLYYHVFRRISKLES
SEQ ID No 31 - FRB with T to L substitution at 2098
which allows binding to AP21967
MASRILWHEMWHEGLEEASRLYFGERNVKGMFEVLEPLHAMMERGPQTLKE
TSFNQAYGRDLMEAQEWCRKYMKSGNVKDLLQAWDLYYHVFRRISKLES
SEQ ID No 32 - FRB segment of mTOR with T to H
substitution at 2098 and to W at F at residue 2101
of the full mTOR which binds Rapamycin with reduced
affinity to wild type
MASRILWHEMWHEGLEEASRLYFGERNVKGMFEVLEPLHAMMERGPQTLKE
TSFNQAYGRDLMEAQEWORKYMKSGNVKDLHQAFDLYYHVFRRISKLES
SEQ ID No 33 - FRB segment of mTOR with K to P
substitution at residue 2095 of the full mTOR which
binds Rapamycin with reduced affinity
MASRILWHEMWHEGLEEASRLYFGERNVKGMFEVLEPLHAMMERGPQTLKE
TSFNQAYGRDLMEAQEWORKYMKSGNVPDLTQAWDLYYHVFRRISKLES

Variant sequences may have at least 80%, 85%, 90%, 95%, 98% or 99% sequence identity to SEQ ID No. 29 to 33, provided that the sequences provide an effective dimerization system. That is, provided that the sequences facilitate co-localisation of membrane tethering component and the signal dampening component at the call membrane.

The “wild-type” FRB domain shown as SEQ ID No, 30 comprises amino acids 2025-2114 of human mTOR. Using the amino acid numbering system of human mTOR, the FRB sequence may comprise an amino acid substitution at one of more of the following positions: 2095, 2098, 2101.

The variant FRB may comprise one of the following amino acids at positions 2095, 2098 and 2101:

2095: K, P, T or A

2098: T, L, H or F

2101: W or F

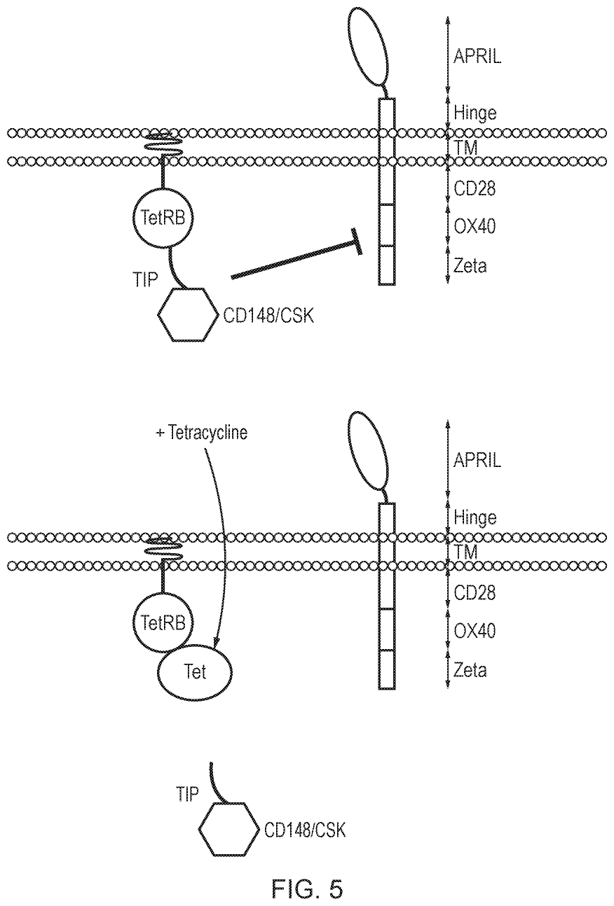

Bayle et al (Chem Bio; 2006; 13; 99-107) describe the following FRB variants, annotated according to the amino acids at positions 2095, 2098 and 2101 (see Table 1): KTW, PLF, KLW, PLW, TLW, ALW, PTF, ATF, TTF, KLF, PLF, TLF, ALF, KTF, KHF, KFF, KLF. These variants are capable of binding rapamycin and rapalogs to varying extents, as shown in Table 1 and FIG. 5A of Bayle et al. The MTC or SDC of the cell of the invention may comprise one of these FRB variants.

In order to prevent rapamycin binding and inactivating endogenous mTOR, the surface of rapamycin which contacts FRB may be modified. Compensatory mutation of the FRB domain to form a burface that accommodates the “bumped” rapamycin restores dimerizing interactions only with the FRB mutant and not to the endogenous mTOR protein.

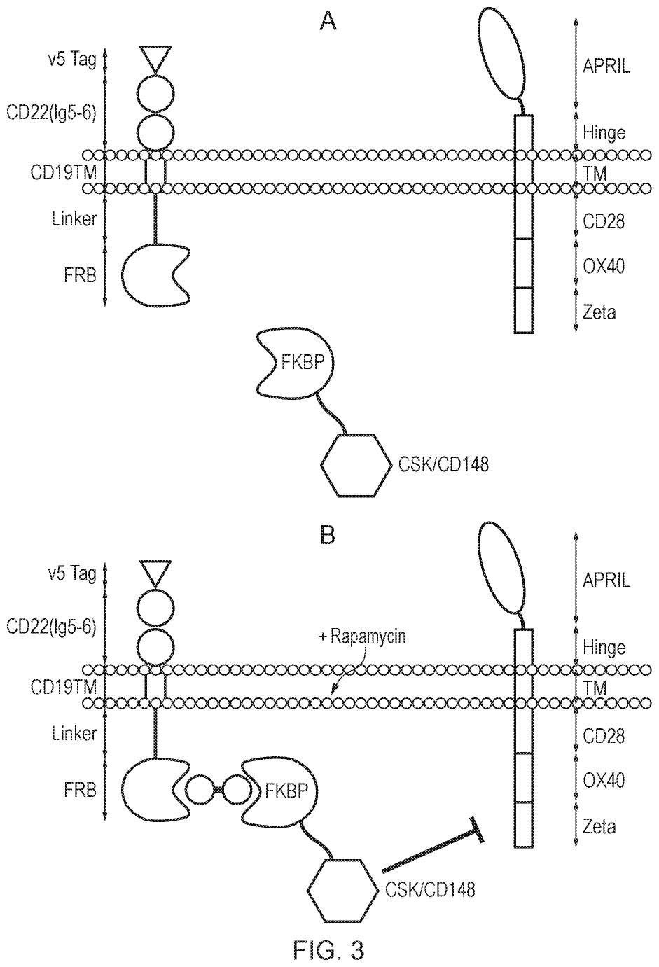

Bayle et al. (as above) describe various rapamycin analogs, or “rapalogs” and their corresponding modified FRB binding domains. For example: C-20-methyllydrapamycin (MaRap), C16(S)-Butylsulfonamidorapamycin (C16-BS-Rap) and C16-(S)-7-methylindolerapamycin (AP21976/C16-AiRap), as shown in FIG. 3, in combination with the respective complementary binding domains for each. Other rapamycins/rapalogs include sirolimus and tacrolimus.

Agent-Disrupted Dimerisation

In a third embodiment, the first and second dimerization domains are capable of dimerising only in the absence of an agent. In this embodiment, dimerization between the first and second dimerization domains is disrupted by the presence of an agent. The agent therefore causes the membrane tethering component and the signal dampening component to dissociate.

The agent may be a molecule, for example a small molecule, which is capable of specifically binding to the first dimerisation domain or the second dimerisation domain at a higher affinity than the binding between the first dimerisation domain and the second dimerisation domain.

For example, the binding system may be based on a peptide:peptide binding domain system. The first or second binding domain may comprise the peptide binding domain and the other binding domain may comprise a peptide mimic which binds the peptide binding domain with lower affinity than the peptide. The use of peptide as agent disrupts the binding of the peptide mimic to the peptide binding domain through competitive binding. The peptide mimic may have a similar amino acid sequence to the “wild-type” peptide, but with one of more amino acid changes to reduce binding affinity for the peptide binding domain.

In this embodiment, the agent may bind the first binding domain or the second binding domain with at least 10, 20, 50, 100, 1000 or 10000-fold greater affinity than the affinity between the first binding domain and the second binding domain.

Small molecules agents which disrupt protein-protein interactions have long been developed for pharmaceutical purpose (reviewed by Vassilev et al; Small-Molecule Inhibitors of Protein-Protein Interactions ISBN: 978-3-642-17082-9). The proteins or peptides whose interaction is disrupted (or relevant fragments of these proteins) can be used as the first and/or second dimerisation domains and the small molecule may be used as the agent.

A list of proteins/peptides whose interaction is disruptable using an agent such as a small molecule is given in Table 2. These disputable protein-protein interactions (PPI) may be used in the dampenable CAR system of the present invention. Further information on these PPIs is available from White et al 2008 (Expert Rev, Mol. Med. 10:e8).

TABLE 2

| Interacting Protein 1 | Interacting Protein 2 | Inhibitor of PPI |
|---|---|---|
| p53 | MDM2 | Nutlin |
| Anti-apoptotic | Apoptotic Bcl2 member | GX015 and ABT-737 |
| Bcl2 member | | |
| Caspase-3, -7 or -9 | X-linked inhibitor of | DIABLO and DIABLO |
| | apoptosis protein (XIAP) | mimetics |
| RAS | RAF | Furano-indene |
| | | derivative |
| FR2-7 | PD2 domain of DVL | FJ9 |
| T-cell factor (TCF) | Cyclic AMP response | ICG-001 |
| | element binding protein | |
| | (CBP) | |

The Tet Repressor (TetR) System

Other small molecule systems for controlling the co-localization of peptides are known in the art, for example the Tet repressor (TetR), TetR interacting protein (TiP), tetracycline system.

The Tet operon is a well-known biological operon which has been adapted for use in mammalian cells. The TetR binds tetracycline as a homodimer and undergoes a conformational change which then modulates the DNA binding of the TetR molecules. Klotzsche et al. (as above), described a phage-display derived peptide which activates the TetR. This protein (TetR interacting protein/TiP) has a binding site in TetR which overlaps, but is not identical to, the tetracycline binding site. Thus TiP and tetracycline compete for binding of TetR.

In the cell of the invention the first dimerisation domain of the membrane tethering component may be TetR or TiP, and the second dimerisation domain of the signal dampening component may be the corresponding, complementary binding partner.

The amino acid sequences of TetR and TiP are shown below as SEQ ID NO: 34 or SEQ ID NO: 35 respectively.

SEQ ID NO: 34 - TetR
MSRLDKSKVINSALELLNEVGIEGLTTRKLAQKLGVEQPTLYWHVKNKRAL
LDALAIEMLDRHHTHFCPLEGESWQDFLRNNAKSFRCALLSHRDGAKVHLG
TRPTEKQYETLENQLAFLCQQGFSLENALYALSAVGH
SEQ ID NO: 35 - TiP
MWTWNAYAFAAPSGGGS

Where the first and second dimerisation domains are TetR or TiP, the agent may be tetracycline, doxycycline, minocycline or an analogue thereof. An analogue refers to a variant of tetracycline, doxycycline or minocycline which retains the ability to specifically bind to TetR.

Streptavidin-Binding Epitope

The first or second dimerisation domain may comprise one or more streptavidin-binding epitope(s). The other binding domain may comprise a biotin mimic.

Streptavidin is a 52.8 kDa protein from the bacterium Streptomyces avidinii. Streptavidin homo-tetramers have a very high affinity for biotin (vitamin B7 or vitamin H), with a dissociation constant (Kd)˜10⁻¹⁵ M. The biotin mimic has a lower affinity for streptavidin than wild-type biotin, so that biotin itself can be used as the agent to disrupt or prevent heterodimerisation between the streptavidin domain and the biotin mimic domain. The biotin mimic may bind streptavidin with for example with a Kd of 1 nM to 100 uM.

The ‘biotin mimic’ domain may, for example, comprise a short peptide sequence (for example 6 to 20, 6 to 18, 8 to 18 or 8 to 15 amino acids) which specifically binds to streptavidin.

The biotin mimic may comprise a sequence as shown in Table 1.

TABLE 1
Biotin mimicking peptides.

| name | Sequence | affinity |
|---|---|---|
| Long nanotag | DVEAWLDERVPLVET | 3.6 nM |
| | (SEQ ID NO: 36) | |
| Short nanotag | DVEAWLGAR | 17 nM |
| | (SEQ ID NO: 37) | |
| Streptag | WRHPQFGG | |
| | (SEQ ID NO: 38) | |
| streptagII | WSHPQFEK | 72 uM |
| | (SEQ ID NO: 39) | |
| SBP-tag | MDEKTTGWRGGHVVEGLAG | 2.5 nM |
| | ELEQLRARLEHHPQGQREP | |
| | (SEQ ID NO: 40) | |
| ccstreptag | CHPQGPPC | 230 nM |
| | (SEQ ID NO: 41) | |
| flankedccstreptag | AECHPQGPPCIEGRK | |
| | (SEQ ID NO: 42) | |

The biotin mimic may be selected from the following group: StreptagII, Flankedccstreptag and ccstreptag.

The streptavidin domain may comprise streptavidin having the sequence shown as SEQ ID No. 43 or a fragment or variant thereof which retains the ability to bind biotin.

Full length Streptavidin has 159 amino acids. The N and C termini of the 159 residue full-length protein are processed to give a shorter ‘core’ streptavidin, usually composed of residues 13-139; removal of the N and C termini is necessary for the high biotin-binding affinity.

The sequence of “core” streptavidin (residues 13-139) is shown as SEQ ID No. 43.

SEQ ID No. 43
EAGITGTWYNQLGSTFIVTAGADGALTGTYESAVGNAESRYVLTGRYDSA
PATDGSGTALGWTVAWKNNYRNAHSATTWSGQYVGGAEARINTQWLLTSG
TTEANAWKSTLVGHDTFTKVKPSAAS

Streptavidin exists in nature as a homo-tetramer. The secondary structure of a streptavidin monomer is composed of eight antiparallel β-strands, which fold to give an antiparallel beta barrel tertiary structure. A biotin binding-site is located at one end of each β-barrel. Four identical streptavidin monomers (i.e. four identical β-barrels) associate to give streptavidin's tetrameric quaternary structure. The biotin binding-site in each barrel consists of residues from the interior of the barrel, together with a conserved Trp120 from neighbouring subunit. In this way, each subunit contributes to the binding site on the neighbouring subunit, and so the tetramer can also be considered a dimer of functional dimers.

The streptavidin domain may consist essentially of a streptavidin monomer, dimer or tetramer.

A variant streptavidin sequence may have at least 70, 80, 90, 95 or 99% identity to SEQ ID No. 43 or a functional portion thereof. Variant streptavidin may comprise one or more of the following amino acids, which are involved in biotin binding: residues Asn23, Tyr43, Ser27, Ser45, Asn49, Ser88, Thr90 and Asp128. Variant streptavidin may, for example, comprise all 8 of these residues. Where variant streptavidin is present in the binding domain as a dimer or teTramer, it may also comprise Trp120 which is involved in biotin binding by the neighbouring subunit.

Destabilisation Domain

The signal dampening component also comprises a destabilisation domain. The presence of a destabilisation domain in a protein means that the stability of the protein is dependent on the presence of an agent, such as a small molecule. When the agent is present, the domain is stabilised and the protein is expressed as normal. In the absence of the agent, the domain is unstable and causes the protein to be unstable such that it collapses and/or is degraded.

The destabilisation domain may comprise a degradation prone mutant of the FK506-rapamycin binding (FRB) domain. For example FRB with a T2098L point mutation is unstable in the absence of rapamycin, but stable when rapamycin is added.

The SDC of the invention may comprise a destabilisation domain which is a degradation prone-mutant of FRB, such as one of the mutants described in Stankunas et al (2007; ChemBioChem, 8: 1162-1169).

The destabilisation domain may comprise the amino acid sequence shown as SEQ ID No. 57

SEQ ID No. 57 (FRBmut)
ASRILWHEMWHEGLEEASRLYFGERNVKGMFEVLEPLHAMMERGPQTLKE
TSFNQAYGRDLMEAQEWCRKYMKSGNVPDLLQAFDLYYHVFRRISKLEY

Mutant versions of FKBP12 which are degraded upon expression have also been described (Banaszynski et al (2006) Cell 126:995-1004). Addition of a synthetic ligand which binds the destabilisation domain shields it from degradation, allowing the protein which comprises the destabilisation domain to be expressed.

The destabilisation domain may comprise a mutant of the FKBP12 F36V sequence shown as SEQ ID NO. 58 with one of the following point mutations: F15S, V24A, H25R, E60G, L106P

SEQ ID No. 58 (FKBP12 F36V)
GVQVETISPGDGRTFPKRGQTCVVENTGMLEDGKKVDSSRDRNKPFKFML
GKQEVIRGWEEGVAQMSVGQRAKLTISPDYAYGATGHPGIIPPHATLVFD
VELLKLE

The destabilisation domain may comprise FKBP12 L106P having the amino acid sequence shon as SEQ ID NO. 59

SEQ ID No. 59 (FKBP12 L106P)
GVQVETISPGDGRTFPKRGQTCVVHYTGMLEDGKKVDSSRDRNKPFKFML
GKQEVIRGWEEGVAQMSVGQRAKLTISPDYAYGATGHPGIIPPHATLVFD
VELLKPE

The ligand which stabilises the mutant versions of FKPB12 is a derivative of SLF* in which the carboxylic acid is replaced with a morpholine group. This molecule is known as the morpholine-containing ligand Shield-1 (Shld1) (Banaszynski et al (2006) as above).

Mutants of the E. coli dihydrofolate reductase (ecDHFR) have also been engineered to be degraded, and it has been shown that when this destabilizing domain is fused to a protein of interest, its instability is conferred to the fused protein resulting in rapid degradation of the entire fusion protein (Iwamoto et al (2010) 17:981-988). It was shown that the small-molecule ligand trimethoprim (TMP) stabilizes the destabilizing domain in a rapid, reversible, and dose-dependent manner, and protein levels in the absence of TMP are barely detectable.

The SDC of the present invention may comprise a mutant DHFR sequence, such as DHFR F103L; R12Y/Y100I or N18T/A19V,

Nucleic Acid Construct

The present invention provides nucleic acid sequences encoding one or more of a chimeric antigen receptor (CAR); a membrane-tethering component (MTC); and a signal-dampening component (SDC) as defined above.

A nucleic acid sequence encoding the CAR may have the following structure:

AgB-spacer-TM-endo

in which

AgB is a nucleic acid sequence encoding an antigen-binding domain;

spacer is a nucleic acid sequence encoding a spacer;

TM1 is a nucleic acid sequence encoding a transmembrane domain;

endo is a nucleic acid sequence encoding an intracellular signalling domain.

A nucleic acid encoding the membrane tethering component may have the following structure:

MLD-DD1; or

DD1-MLD

in which

MLD is a nucleic acid sequence encoding a membrane localisation domain; and

DD1 is a nucleic acid sequence encoding a first dimerization domain.

A nucleic acid sequence encoding the signal dampening component may have the following structure:

SDD-DD2; or

DD2-SDD

in which

SDD is a nucleic acid sequence encoding a signal dampening domain; and

DD2 is a nucleic acid sequence encoding a second dimerization domain.

The present invention provides a nucleic acid construct which comprises:
(i) a first nucleic acid sequence which encodes a chimeric antigen receptor (CAR);
(ii) a second nucleic acid sequence which encodes a membrane-tethering component (MTC); and
(iii) a third nucleic acid sequence which encodes a signal-dampening component (SDC).

The first, second and third nucleic acid sequences may be in any order in the construct, i.e.:

CAR-MTC-SDC;

CAR-SDC-MTC;

MTC-CAR-SDC;

MTC-SDC-CAR;

SDC-CAR-MTC; or

SDC-MTC-CAR.

In the construct, the nucleic acid sequences may be connected by sequences enabling co-expression of the CAR, MTC and SDC as separate polypeptides. For example, the nucleic acid may encode a cleavage site between two of the components; or two cleavage sites, enabling the production of all three components as discrete polypeptides. The cleavage site may be self-cleaving, such that when the compound polypeptide is produced, it is immediately cleaved into the separate components without the need for any external cleavage activity.

Various self-cleaving sites are known, including the Foot-and-Mouth disease virus (FMDV) 2a self-cleaving peptide, which may have one of the following sequences:

SEQ ID NO: 44
RAEGRGSLLTCGDVEENPGP
or
SEQ ID NO: 45
QCTNYALLKLAGDVESNPGP

The co-expressing sequence may be an internal ribosome entry sequence (IRES), The co-expressing sequence may be an internal promoter.

The nucleic acid construct may, for example, encode the following:

SFGmR.V5_tag-CD22(2Ig)-CD19tm-RL-FRB-2A-FKBP12-L-CD148endo-2A-CAR

in which

“SFGmR” is a signal peptide derived from murine Ig kappa chain V-III region, having the sequence:

| | |
|---|---|
| METDTLLLWVLLLWVPGSTG | (SEQ ID No. 46) |

“V5 tag-” is a is a Linker-V5 tag-Linker, having the sequence:

| | |
|---|---|
| DSSGKPIPNPLLGLDSSGGGGSA | (SEQ ID No. 47) |

“CD22(2Ig)” are the two most membrane proximal Ig domains from human CD22, having the sequence:

(SEQ ID No. 48)
PRDVRVRKIKPLSEIHSGNSVSLQCDFSSSHPKEVQFFWEKNGRLLGKES
QLNFDSISPEDAGSYSCWVNNSIGQTASKAWTLEVLYAPRRLRVSMSPGD
QVMEGKSATLTCESDANPPVSHYTWFDWNNQSLPYHSQKLRLEPVKVQHS
GAYWCQGTNSVGKGRSPLSTLTVYYSPETIGRR

“CD19tm” is a sequence comprising the CD19 transmembrane sequence and truncated CD19 endodomain, having the sequence:

(SEQ ID No. 49)
AVTLAYLIFCLCSLVGILHLQRALVLRRKRKRMTDPTRR

“RL” is a Rigid Linker having the sequence:

(SEQ ID No. 50)
LEAEAAAKEAAAKEAAAKEAAAKALEAEAAAKEAAAKEAAAKEAAAKALE
SGGGSASR

“FRB” is an FRB domain having the sequence:

(SEQ ID No. 51)
ILWHEMWHEGLEEASRLYFGERNVKGMFEVLEPLHAMMERGPQTLKETSF
NQAYGRDLMEAQEWCRKYMKSGNVKDLLQAWDLYYHVFRRISKLEYSAS

“2A” is an FMDV 2A peptide having the sequence:

| | |
|---|---|
| EGRGSLLTCGDVEENPGP | (SEQ ID No. 52) |

“FKBP12” is an FKBP12 domain having the sequence:

(SEQ ID No. 53)
GVQVETISPGDGRTFPKRGQTCVVHYTGMLEDGKKFDSSRDRNKPFKFML
GKQEVIRGWEEGVAQMSVGQRAKLTISPDYAYGATGHPGIIPPHATLVFD
VELLKLE

“L” is a linker having the sequence:

| | |
|---|---|
| SGGGSG | (SEQ ID No. 61) |

“CD148endo” is a CD148 endodomain having the sequence:

(SEQ ID No. 4)
RKKRKDAKNNEVSFSQIKPKKSKLIRVENFEAYFKKQQADSNCGFAEEYE
DLKLVGISQPKYAAELAENRGKNRYNNVLPYDISRVKLSVQTHSTDDYIN
ANYMPGYHSKKDFIATQGPLPNTLKDFWRMVWEKNVYAIIMLTKCVEQGR
TKCEEYWPSKQAQDYGDITVAMTSEIVLPEWTIRDFTVKNIQTSESHPLR
QFHFTSWPDHGVPDTTDLLINFRYLVRDYMKQSPPESPILVHCSAGVGRT
GTFIAIDRLIYQIENENTVDVYGIVYDLRMHRPLMVQTEDQYVFLNQCVL
DIVRSQKDSKVDLIYQNTTAMTIYENLAPVTTFGKTNGYIA

“2A” is an FMDV 2A peptide having the sequence:

(SEQ ID No. 52)
EGRGSLLTCGDVEENPGP

“CAR” is a second generation anti-CD19 CAR having a CD28-Zeta endodomain, the CAR having the sequence:

(SEQ ID No. 60)
METDTLLLWVLLLWVPGSTGDIQMTQTTSSLSASLGDRVTISCRASQDIS
KYLNWYQQKPDGTVKLLIYHTSRLHSGVPSRFSGSGSGTDYSLTISNLEQ
EDIATYFCQQGNTLPYTFGGGTKLEITKAGGGGSGGGGSGGGGSGGGGSE
VKLQESGPGLVAPSQSLSVTCTVSGVSLPDYGVSWIRQPPRKGLEWLGVI
WGSETTYYNSALKSRLTIIKDNSKSQVFLKMNSLQTDDTAIYYCAKHYYY
GGSYAMDYWGQGTSVTVSSDPTTTPAPRPPTPAPTIASQPLSLRPEACRP
AAGGAVHTRGLDFACDIYIWAPLAGTCGVLLLSLVITLYCRKKRSRSKRS
RLLHSDYMNMTPRRPGPTRKHYQPYAPPRDFAAYRSRVKFSRSADAPAYQ
QGQNQLYNELNLGRREEYDVLDKRRGRDPEMGGKPRRKNPQEGLYNELQK
DKMAEAYSEIGMKGERRRGKGHDGLYQGLSTATKDTYDALHMQALPPR

When this construct is expressed in a cell, rapamycin or an analogue thereof can be used to induce dimerization of the FRB-containing membrane-tethering component with the FKBP12-containing signal dampening component, causing dampening of CAR-mediated cell signalling (see FIG. 3).

As an alternative example, the nucleic acid construct may encode the following:

SFG.TIP-L(16aa)-CD148endo-2A-V5-tag-CD22(2Ig)-CD19tm-RL-TetRB-2A-CAR

in which

“SFG” is a signal peptide derived from murine Ig kappa chain V-III region, having the sequence:

(SEQ ID No. 46)
METDTLLLWVLLLWVPGSTG

“TIP” is a TetR interacting peptide having the sequence:

(SEQ ID No. 54)
MWTWNAYAFAAP

“L” is a Linker having the sequence:

(SEQ ID No. 55)
SGGGGSGGGGSGGGGS

“CD148endo” is a CD148 endodomain having the sequence:

(SEQ ID No. 4)
RKKRKDAKNNEVSFSQIKPKKSKLIRVENFEAYFKKQQADSNCGFAEEYE
DLKLVGISQPKYAAELAENRGKNRYNNVLPYDISRVKLSVQTHSTDDYIN
ANYMPGYHSKKDFIATQGPLPNTLKDFWRMVWEKNVYAIIMLTKCVEQGR
TKCEEYWPSKQAQDYGDITVAMTSEIVLPEWTIRDFTVKNIQTSESHPLR
QFHFTSWPDHGVPDTTDLLINFRYLVRDYMKQSPPESPILVHCSAGVGRT
GTFIAIDRLIYQIENENTVDVYGIVYDLRMHRPLMVQTEDQYVFLNQCVL
DIVRSQKDSKVDLIYQNTTAMTIYENLAPVTTFGKTNGYIA

“2A” is an FMDV 2A peptide having the sequence:

(SEQ ID No. 52)
EGRGSLLTCGDVEENPGP

“V5_tag-” is a is a Linker-V5 tag-Linker, having the sequence:

(SEQ ID No. 47)
DSSGKPIPNPLLGLDSSGGGGSA

“CD22(2Ig)” are the two most membrane proximal Ig domains from human CD22, having the sequence:

(SEQ ID No. 48)
PRDVRVRKIKPLSEIHSGNSVSLQCDFSSSHPKEVQFFWEKNGRLLGKES
QLNFDSISPEDAGSYSCWVNNSIGQTASKAWTLEVLYAPRRLRVSMSPGD
QVMEGKSATLTCESDANPPVSHYTWFDWNNQSLPYHSQKLRLEPVKVQHS
GAYWCQGTNSVGKGRSPLSTLTVYYSPETIGRR

“CD19tm” is a sequence comprising the CD19 transmembrane sequence and truncated CD19 endodomain, having the sequence:

(SEQ ID No. 49)
AVTLAYLIFCLCSLVGILHLQRALVLRRKRKRMTDPTRR

“RL” is a Rigid Linker having the sequence:

(SEQ ID No. 50)
LEAEAAAKEAAAKEAAAKEAAAKALEAEAAAKEAAAKEAAAKEAAAKALE
SGGGSASR

TetRB is a Tet repressor B protein having the sequence:

(SEQ ID No. 56)
MSRLDKSKVINSALELLNEVGIEGLTTRKLAQKLGVEQPTLYWHVKNKRA
LLDALAIEMLDRHHTHFCPLEGESWQDFLRNNAKSFRCALLSHRDGAKVH
LGTRPTEKQYETLENQLAFLCQQGFSLENALYALSAVGHFTLGCVLEDQE
HQVAKEERETPTTDSMPPLLRQAIELFDHQGAEPAFLFGLELIICGLEKQ
LKCESGS

“2A” is an FMDV 2A peptide having the sequence:

(SEQ ID No. 52)
EGRGSLLTCGDVEENPGP

“CAR” is a second generation anti-CD19 CAR having a CD28-Zeta endodomain, the CAR having the sequence:

(SEQ ID No. 60)
METDTLLLWVLLLWVPGSTGDIQMTQTTSSLSASLGDRVTISCRASQDIS
KYLNWYQQKPDGTVKLLIYHTSRLHSGVPSRFSGSGSGTDYSLTISNLEQ
EDIATYFCQQGNTLPYTFGGGTKLEITKAGGGGSGGGGSGGGGSGGGGSE
VKLQESGPGLVAPSQSLSVTCTVSGVSLPDYGVSWIRQPPRKGLEWLGVI
WGSETTYYNSALKSRLTIIKDNSKSQVFLKMNSLQTDDTAIYYCAKHYYY
GGSYAMDYWGQGTSVTVSSDPTTTPAPRPPTPAPTIASQPLSLRPEACRP
AAGGAVHTRGLDFACDIYIWAPLAGTCGVLLLSLVITLYCRKKRSRSKRS
RLLHSDYMNMTPRRPGPTRKHYQPYAPPRDFAAYRSRVKFSRSADAPAYQ
QGQNQLYNELNLGRREEYDVLDKRRGRDPEMGGKPRRKNPQEGLYNELQK
DKMAEAYSEIGMKGERRRGKGHDGLYQGLSTATKDTYDALHMQALPPR

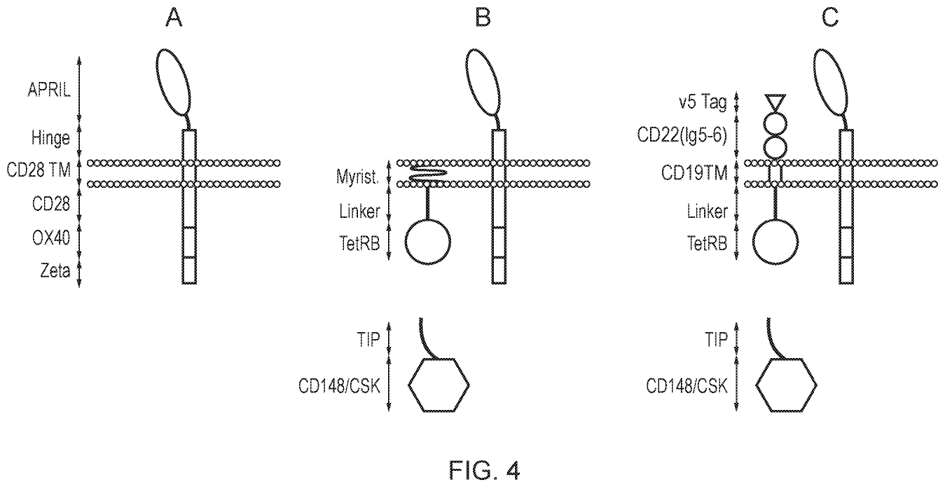

When this construct is expressed in a cell tetracyclin or an analogue thereof can be used to disrupt dimerization of the TetRB-containing membrane-tethering component with the TiP-containing signal dampening component. This releases the CAR from the dampening effect of the signal damening domain, meaning that CAR-mediated signalling can occur (see FIGS. 4 to 6)

As used herein, the terms “polynucleotide”, “nucleotide”, and “nucleic acid” are intended to be synonymous with each other.

It will be understood by a skilled person that numerous different polynucleotides and nucleic acids can encode the same polypeptide as a result of the degeneracy of the genetic code. In addition, it is to be understood that skilled persons may, using routine techniques, make nucleotide substitutions that do not affect the polypeptide sequence encoded by the polynucleotides described here to reflect the codon usage of any particular host organism in which the polypeptides are to be expressed.

Nucleic acids according to the invention may comprise DNA or RNA. They may be single-stranded or double-stranded. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the use as described herein, it is to be understood that the polynucleotides may be modified by any method available in the art. Such modifications may be carried out in order to enhance the in vivo activity or life span of polynucleotides of interest.

The terms “variant”, “homologue” or “derivative” in relation to a nucleotide sequence include any substitution of, variation of, modification of, replacement of, deletion of or addition of one (or more) nucleic acid from or to the sequence.

The present invention also provides a kit comprising a first nucleic acid sequence encoding a chimeric antigen receptor (CAR); a second nucleic acid sequence encoding a membrane-tethering component (MTC); and a third nucleic acid sequence encoding a signal-dampening component (SDC).

Vector

The present invention also provides a vector, or kit of vectors which comprises one or more nucleic acid sequence(s) of the invention. Such a vector may be used to introduce the nucleic acid sequence(s) into a host cell so that it expresses the CAR, MTC and/or SDC as defined above.

The vector may, for example, be a plasmid or a viral vector, such as a retroviral vector or a lentiviral vector, or a transposon based vector or synthetic mRNA.

The vector may be capable of transfecting or transducing a T cell or a NK cell.

Cell

The present invention relates to a cell which comprises a dampenable CAR system.

The cell may comprise a nucleic acid or a vector of the present invention.

The cell may be an immune cell, such as a cytolytic immune cell. Cytolytic immune cells can be T cells or T lymphocytes which are a type of lymphocyte that play a central role in cell-mediated immunity. They can be distinguished from other lymphocytes, such as B cells and natural killer cells (NK cells), by the presence of a T-cell receptor (TCR) on the cell surface. There are various types of T cell, as summarised below.

Helper T helper cells (TH cells) assist other white blood cells in immunologic processes, including maturation of B cells into plasma cells and memory B cells, and activation of cytotoxic T cells and macrophages. TH cells express CD4 on their surface. TH cells become activated when they are presented with peptide antigens by MHC class II molecules on the surface of antigen presenting cells (APCs). These cells can differentiate into one of several subtypes, including TH1, TH2, TH3, TH17, Th9, or TFH, which secrete different cytokines to facilitate different types of immune responses.

Cytolytic T cells (TC cells, or CTLs) destroy virally infected cells and tumor cells, and are also implicated in transplant rejection. CTLs express the CD8 at their surface. These cells recognize their targets by binding to antigen associated with MHC class I, which is present on the surface of all nucleated cells. Through IL-10, adenosine and other molecules secreted by regulatory T cells, the CD8+ cells can be inactivated to an anergic state, which prevent autoimmune diseases such as experimental autoimmune encephalomyelitis.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory T cells comprise three subtypes: central memory T cells (TCM cells) and two types of effector memory T cells (TEM cells and TEMRA cells). Memory cells may be either CD4+ or CD8+. Memory T cells typically express the cell surface protein CD45RO.

Regulatory T cells (Treg cells), formerly known as suppressor T cells, are crucial for the maintenance of immunological tolerance. Their major role is to shut down T cell-mediated immunity toward the end of an immune reaction and to suppress auto-reactive T cells that escaped the process of negative selection in the thymus.

Two major classes of CD4+ Treg cells have been described—naturally occurring Treg cells and adaptive Treg cells.

Naturally occurring Treg cells (also known as CD4+CD25+FoxP3+ Treg cells) arise in the thymus and have been linked to interactions between developing T cells with both myeloid (CD11c+) and plasmacytoid (CD123+) dendritic cells that have been activated with TSLP. Naturally occurring Treg cells can be distinguished from other T cells by the presence of an intracellular molecule called FoxP3. Mutations of the FOXP3 gene can prevent regulatory T cell development, causing the fatal autoimmune disease IPEX.

Adaptive Treg cells (also known as Tr1 cells or Th3 cells) may originate during a normal immune response.

Natural Killer Cells (or NK cells) are a type of cytolytic cell which form part of the innate immune system. NK cells provide rapid responses to innate signals from virally infected cells in an MHC independent manner

NK cells (belonging to the group of innate lymphoid cells) are defined as large granular lymphocytes (LGL) and constitute the third kind of cells differentiated from the common lymphoid progenitor generating B and T lymphocytes. NK cells are known to differentiate and mature in the bone marrow, lymph node, spleen, tonsils and thymus where they then enter into the circulation.

The CAR-expressing cells of the invention may be any of the cell types mentioned above.

CAR-expressing cells, such as T or NK cells may either be created ex vivo either from a patient's own peripheral blood (1st party), or in the setting of a haematopoietic stem cell transplant from donor peripheral blood (2nd party), or peripheral blood from an unconnected donor (3rd party).

Alternatively, CAR-expressing cells may be derived from ex vivo differentiation of inducible progenitor cells or embryonic progenitor cells to T cells. Alternatively, an immortalized T-cell line which retains its lytic function and could act as a therapeutic may be used.

In all these embodiments, CAR cells are generated by introducing DNA or RNA coding for the receptor component and signalling component by one of many means including transduction with a viral vector, transfection with DNA or RNA.

The CAR cell of the invention may be an ex vivo T or NK cell from a subject. The T or NK cell may be from a peripheral blood mononuclear cell (PBMC) sample. T or NK cells may be activated and/or expanded prior to being transduced with nucleic acid encoding the molecules providing the CAR system according to the first aspect of the invention, for example by treatment with an anti-CD3 monoclonal antibody.

The cell of the invention may be made by:
(i) isolation of a cell-containing sample from a subject or other sources listed above; and
(ii) transduction or transfection of the cells with one or more a nucleic acid sequence(s) or nucleic acid construct as defined above.

The cells may then by purified, for example, selected on the basis of expression of the antigen-binding domain of the antigen-binding polypeptide.

Pharmaceutical Composition

The present invention also relates to a pharmaceutical composition containing a plurality of cells of the invention. The pharmaceutical composition may additionally comprise a pharmaceutically acceptable carrier, diluent or excipient. The pharmaceutical composition may optionally comprise one or more further pharmaceutically active polypeptides and/or compounds. Such a formulation may, for example, be in a form suitable for intravenous infusion.

Method of Treatment

The present invention provides a method for treating and/or preventing a disease which comprises the step of administering the cells of the present invention (for example in a pharmaceutical composition as described above) to a subject.

A method for treating a disease relates to the therapeutic use of the cells of the present invention. In this respect, the cells may be administered to a subject having an existing disease or condition in order to lessen, reduce or improve at least one symptom associated with the disease and/or to slow down, reduce or block the progression of the disease.

The method for preventing a disease relates to the prophylactic use of the cells of the present invention. In this respect, the cells may be administered to a subject who has not yet contracted the disease and/or who is not showing any symptoms of the disease to prevent or impair the cause of the disease or to reduce or prevent development of at least one symptom associated with the disease. The subject may have a predisposition for, or be thought to be at risk of developing, the disease.

The method may involve the steps of:
(i) isolating a cell-containing sample;
   (ii) transducing or transfecting such cells with a nucleic acid sequence or vector provided by the present invention;
(iii) administering the cells from (ii) to a subject.

The methods provided by the present invention for treating a disease may involve monitoring the progression of the disease and any toxic activity and administering or removing an agent to inhibit CAR signalling and thereby reduce or lessen any adverse toxic effects.

The methods provided by the present invention for treating a disease may involve monitoring the progression of the disease and monitoring any toxic activity and adjusting the dose of the agent administered to the subject to provide acceptable levels of disease progression and toxic activity.

Monitoring the progression of the disease means to assess the symptoms associated with the disease over time to determine if they are reducing/improving or increasing/worsening.

The present invention also provides a method for controlling the activation of a cell of the invention in a subject, which comprises the step of administering an agent which controls binding or dissociation of the first and second dimerization domains to the subject.

The present invention also provides a method for treating a CAR-associated toxicity in a subject comprising a cell of the invention, which comprises the step of administering an agent which induces binding of the first and second binding domains to the subject.

Toxic activities relate to adverse effects caused by the CAR cells of the invention following their administration to a subject. Toxic activities may include, for example, immunological toxicity, biliary toxicity and respiratory distress syndrome, cytokine release syndrome, macrophage activation syndrome, or a neurotoxicity.

The level of signalling through the CAR, and therefore the level of activation of CAR-expressing cells, may be adjusted by altering the amount of agent present, or the amount of time the agent is present.

Where the agent induces dimerization between the SDC and the MTC, the level of CAR cell activation may be augmented by decreasing the dose of agent administered to the subject or decreasing the frequency of its administration. Conversely, the level of CAR cell activation may be reduced by increasing the dose of the agent, or the frequency of administration to the subject.

Where the agent disrupts dimerization between the SDC and the MTC, the level of CAR cell activation may be augmented by increasing the dose of agent administered to the subject or increasing the frequency of its administration. Conversely, the level of CAR cell activation may be reduced by decreasing the dose of the agent, or the frequency of administration to the subject.

Higher levels of CAR cell activation are likely to be associated with reduced disease progression but increased toxic activities, whilst lower levels of CAR cell activation are likely to be associated with increased disease progression but reduced toxic activities.

The present invention also provides a method for treating and/or preventing a disease in a subject which subject comprises cells of the invention, which method comprises the step of administering an agent to the subject. As such, this method involves administering a suitable agent to a subject which already comprises CAR-expressing cells of the present invention.

As such the dose of agent administered to a subject, or the frequency of administration, may be altered in order to provide an acceptable level of both disease progression and toxic activity. The specific level of disease progression and toxic activities determined to be ‘acceptable’ will vary according to the specific circumstances and should be assessed on such a basis. The present invention provides a method for altering the activation level of the CAR in cells in order to achieve this appropriate level.

The agent may be administered in the form of a pharmaceutical composition. The pharmaceutical composition may additionally comprise a pharmaceutically acceptable carrier, diluent or excipient. The pharmaceutical composition may optionally comprise one or more further pharmaceutically active polypeptides and/or compounds. Such a formulation may, for example, be in a form suitable for intravenous infusion.

The present invention provides a CAR cell of the present invention for use in treating and/or preventing a disease.

The invention also relates to the use of a CAR cell of the present invention in the manufacture of a medicament for the treatment and/or prevention of a disease.

The present invention also provides an agent for dampening CAR-mediated signalling in a cell according to the invention for use in treating and/or preventing a disease.

The present invention also provides an agent for reducing or removing dampening of CAR-mediated signalling in a cell according to the invention for use in treating and/or preventing a disease.

The present invention also provides an agent for use in dampening CAR-mediated signalling in a cell according to the invention.

The present invention also provides an agent for use in reducing or removing dampening of CAR-mediated signalling in a cell according to the invention

The disease to be treated and/or prevented by the methods of the present invention may be an infection, such as a viral infection.

The methods of the invention may also be for the control of pathogenic immune responses, for example in autoimmune diseases, allergies and graft-vs-host rejection.

The methods may be for the treatment of a cancerous disease, such as bladder cancer, breast cancer, colon cancer, endometrial cancer, kidney cancer (renal cell), leukaemia, lung cancer, melanoma, non-Hodgkin lymphoma, pancreatic cancer, prostate cancer and thyroid cancer.

The invention will now be further described by way of Examples, which are meant to serve to assist one of ordinary skill in the art in carrying out the invention and are not intended in any way to limit the scope of the invention.

### EXAMPLES

### Example 1—Functionality of a Rapa-Off Dampening System

A tricistronic construct is expressed as a single transcript having the structure:

SFGm R.V5_tag-CD22(2Ig)-CD19tm-RL-FRB-2A-FKBP12-L-CD148endo-2A-CAR

This self-cleaves at the 2A sites to a FKBP12-containing signal dampening component, a membrane tethering component comprising a transmembrane domain and an intracellular FRB domain, and an anti-CD19 second generation CAR.

The construct is expressed in BW5 cells. SupT1 cells (which are CD19 negative), are engineered to be CD19 positive giving target negative and positive cell lines which are as similar as possible. Primary human T-cells from 3 donors are transduced with two CAR constructs: (I) “Classical” anti-CD19 CAR; (ii) the tri-cistronic “dampenable” CD19 CAR system described above. Non-transduced T-cells and T-cells transduced with the different CAR constructs are challenged 1:1 with either SupT1 cells or SupT1.CD19 cells in the presence of different concentrations of rapamycin. Supernatant is sampled 48 hours after challenge. Supernatant from background (T-cells alone), and maximum (T-cells stimulated with PMA/Ionomycin) ss also sampled. Interferon-gamma is measured in supernatants by ELISA.

Killing of target cells is also demonstrated using a chromium release assay. SupT1 and SupT1.CD19 cells are loaded with ⁵¹Cr and incubated with control and Tet-CAR T-cells for 4 hours in the presence or absence of rapamycin. Lysis of target cells is determined by counting ⁵¹Cr in the supernatant.

As illustrated in FIG. 3, in a “Rapa-Off” dampening system in the absence of rapamycin, the signal dampening component diffuses freely in the cytoplasm, and CAR-mediated signalling can occur. In the presence of rapamycin, the signal dampening component dimerises with the membrane tethering component, bringing CD148 into proximity with the intracellular signalling domain of the CAR, and dampening cell signalling. CAR-mediated activation is therefore “turned down” or “turned off” by the presence of rapamycin

### Example 2—Functionality of a Tet-ON Dampening System

A tricistronic construct is expressed as a single transcript having the structure:

SFG.TIP-L(16aa)-CD148endo-2A-V5-tag-CD22(2Ig)-CD19tm-RL-TetRB-2A-CAR

This self-cleaves at the 2A sites to a TiP-containing signal dampening component, a membrane tethering component comprising a transmembrane domain and an intracellular TetRB domain, and an anti-CD19 second generation CAR.

The construct is expressed in BW5 cells which are then challenged with wild-type SupT1 cells or SupT1 cells engineered to express CD19, in the presence or absence of tetracycline using the methodology described in Example 1.

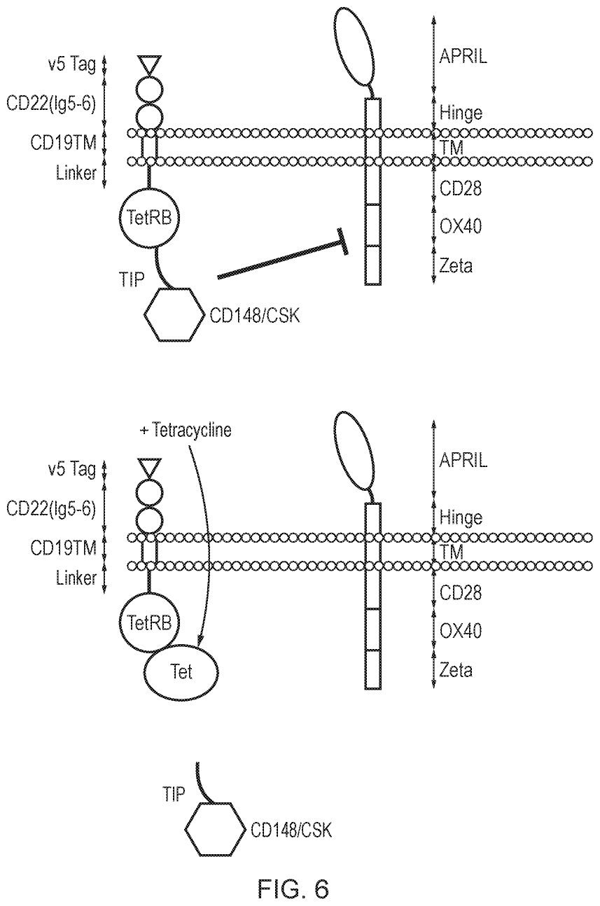

As illustrated in FIGS. 5 and 6, in a “Tet-ON” dampening system in the presence of tetracycline, the signal dampening component diffuses freely in the cytoplasm, and CAR-mediated signalling can occur. In the absence of tetracycline, the signal dampening component dimerises with the membrane tethering component, bringing CD148 into proximity with the intracellular signalling domain of the CAR, and dampening cell signalling. CAR-mediated activation is therefore “turned up” or “turned on” by the presence of tetracycline.

### Example 3—Functionality of a Rapa-Off Damning System with an Anti-BCMA CAR

A tricistronic construct was expressed as a single transcript having the structure:

SFGmR.V5_tag-CD22(2Ig)-TM-FRB-2A-FKBP12-CD148endo-2A-CAR

This self-cleaves at the 2A sites to a FKBP12-containing signal dampening component, a membrane tethering component comprising a transmembrane domain and an intracellular FRB domain, and an anti-BCMA third generation CAR having an antigen binding site based on a proliferation-inducing ligand (APRIL), the natural ligand for BCMA.

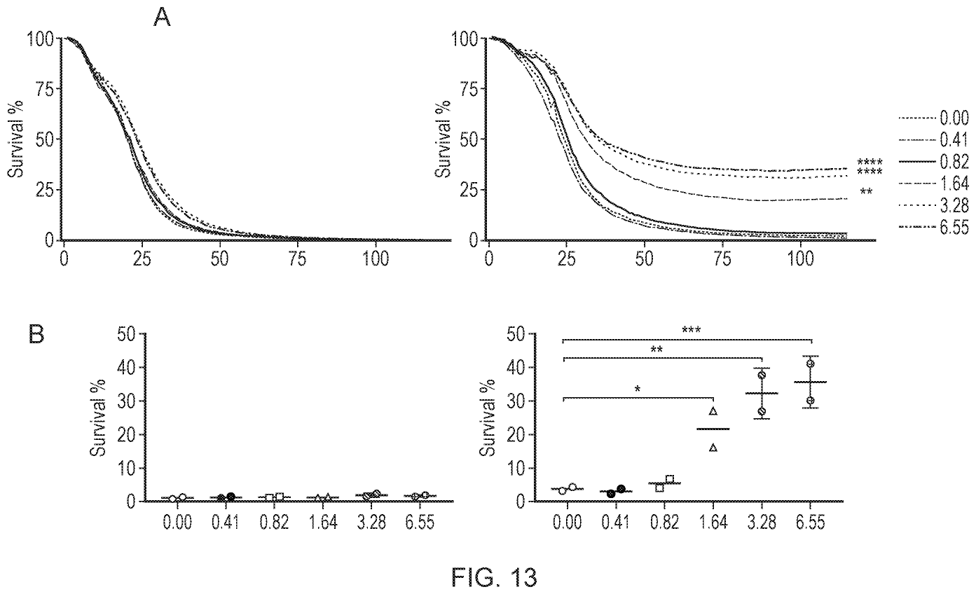

The construct was expressed in BW5 cells. SKOV3 cells, were engineered to be BCMA positive for use as target cells. Primary human T-cells from 2 donors were transduced with two CAR constructs: (i) “Classical” anti-BCMA CAR; (ii) the tri-cistronic “dampenable” BCMA CAR system described above, Non-transduced T-cells and T-cells transduced with the different CAR constructs were challenged 8:1 with SKOV3_BCMA cells in the presence of different concentrations of rapamycin and target cell killing was investigated using an incucyte assay. The results are shown in FIG. 13.

In the presence of rapamycin, killing of target cells by T cells expressing a CAR, membrane tethering component and signal dampening component was found to be significantly inhibited. The inhibition was found to be titratable depending on the concentration of rapamycin. At the 72 hour time point, killing of target cells by T cells expressing a CAR, membrane tethering component and signal dampening component was significantly inhibited at the tested concentrations of Rapamycin above 0.82 μM (FIG. 13B).

### Example 4—Functionality of a Rapa-Off Dampening System with an Anti-CD19 CAR

A tricistronic construct was expressed as a single transcript having the structure:

SFGmR.V5_tag-CD22(2Ig)-TM-FRB-2A-FKBP12-CD148endo-2A-CAR

This self-cleaves at the 2A sites to a FKBP12-containing signal dampening component, a membrane tethering component comprising a transmembrane domain and an intracellular FRB domain, and an anti-CD19 second generation CAR having an antigen binding site based on the antibody fmc63.

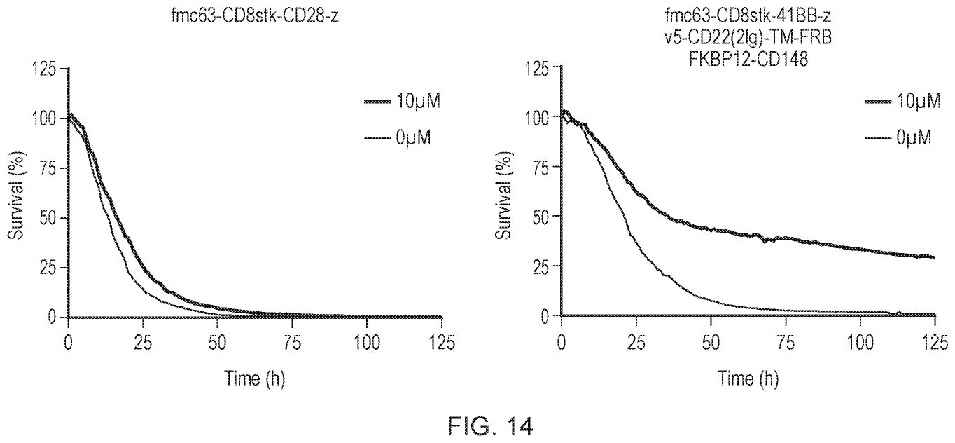

The construct was expressed in BW5 cells. SKOV3 cells were engineered to be Cd19 positive for use as target cells. Primary human T-cells from 2 donors were transduced with two CAR constructs: (i) “Classical” anti-BCMA CAR; (ii) the tri-cistronic “dampenable” anti-CD19 CAR system described above. Non-transduced T-cells and T-cells transduced with the different CAR constructs were challenged 4:1 with SKOV3_BCMA cells in the presence of different concentrations of rapamycin and target cell killing was investigated using an incucyte assay. The results are shown in FIG. 14.

In the presence of rapamycin, killing of target cells by T cells expressing a CAR, membrane tethering component and signal dampening component was significantly inhibited. At 50 hours, the control CAR had almost completely killed the target cells, whereas for T-cells co-expressing the CAR with a dampener, approximately 50% of the target cells were surviving.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments.

Indeed, various modifications of the described modes for carrying out the invention which are obvious to those skilled in molecular biology or related fields are intended to be within the scope of the following claims.

1. A cell which comprises:
(i) a chimeric antigen receptor (CAR) which comprises an antigen binding domain and an intracellular signalling domain;
(ii) a membrane tethering component which comprises a first dimerization domain; and
(iii) a signal-dampening component (SDC) comprising a signal-dampening domain (SDD) and a second dimerization domain which specifically binds the first dimerisation domain of the membrane-tethering component.

2. A cell according to claim 1, wherein the SDD inhibits the intracellular signalling domain of the CAR.

3. A cell according to claim 2, wherein the SDD comprises a phosphatase domain capable of dephosphorylating immunoreceptor tyrosine-based activation motifs (ITAMs).

4.-9. (canceled)

10. A cell according to claim 1, wherein the SDD causes the removal of the intracellular signalling domain of the CAR.

11. A cell according to claim 10, wherein the SDD comprises a protease and the CAR comprises a protease cleavage site.

12. (canceled)

13. A cell according to claim 1 wherein binding of the first and second dimerization domains is controllable by the presence or absence of an agent.

14. A cell according to claim 13, wherein binding of the first and second dimerization domains is induced by the presence of a chemical inducer of dimerisation (CID).

15.-18. (canceled)

19. A cell according to claim 13, wherein the agent disrupts binding of the first and second dimerization domains

20. (canceled)

21. A cell according to claim 1, wherein the membrane tethering component comprises a transmembrane domain or a myristoylation sequence.

22. A nucleic acid construct which comprises:
(i) a first nucleic acid sequence which encodes a chimeric antigen receptor (CAR) which comprises an antigen binding domain and an intracellular signaling domain;
(ii) a second nucleic acid sequence which encodes a membrane-tethering component (MTC) which comprises a first dimerization domain; and
(iii) a third nucleic acid sequence which encodes a signal-dampening component (SDC) comprising a signal-dampening domain (SDD) and a second dimerization domain which specifically binds the first dimerization domain of the membrane-tethering compounds.

23. A kit of comprising:
(i) a first nucleic acid sequence or first vector which encodes a chimeric antigen receptor (CAR) which comprises an antigen binding domain and an intracellular signalling domain;
(ii) a second nucleic acid sequence or second vector which encodes a membrane-tethering component (MTC) which comprises a first dimerization domain; and
(ii) a third nucleic acid sequence or third vector which encodes a signal-dampening component (SDC) comprising a signal-dampening domain (SDD) and a second dimerization domain which specifically binds the first dimerisation domain of the membrane-tethering component.

24. A vector comprising a nucleic acid construct according to claim 22.

25. (canceled)

26. A pharmaceutical composition comprising a plurality of cells according to claim 1.

27. (canceled)

28. A method for treating and/or preventing a disease, which comprises the step of administering a pharmaceutical composition according to claim 26 to a subject.

29. A method according to claim 28, which comprises the following steps:
(i) isolation of a cell-containing sample;
(ii) transduction or transfection of the cells with a nucleic acid construct which comprises:
(i) a first nucleic acid sequence which encodes a chimeric antigen receptor (CAR) which comprises an antigen binding domain and an intracellular signaling domain;
(ii) a second nucleic acid sequence which encodes a membrane-tethering component (MTC) which comprises a first dimerization domain; and
(iii) a third nucleic acid sequence which encodes a signal-dampening component (SDC) comprising a signal-dampening domain (SDD) and a second dimerization domain which specifically binds the first dimerization domain of the membrane-tethering compounds; and
(iii) administering the cells from (ii) to a subject.

30. A method for controlling the activation of a cell according to claim 1 in a subject, which comprises the step of administering an agent which controls binding or dissociation of the first and second dimerization domains to the subject.

31. A method for treating a CAR-associated toxicity in a subject comprising a cell according to claim 1, which comprises the step of administering an agent which induces binding of the first and second binding domains to the subject.

32. A method according to claim 31, where the CAR-associated toxicity is cytokine release syndrome, macrophage activation syndrome, or a neurotoxicity.

33.-34. (canceled)

35. A method for making a cell according to claim 1, which comprises the step of introducing into a cell a nucleic acid construct which comprises:
(i) a first nucleic acid sequence which encodes a chimeric antigen receptor (CAR) which comprises an antigen binding domain and an intracellular signaling domain;
(ii) a second nucleic acid sequence which encodes a membrane-tethering component (MTC) which comprises a first dimerization domain; and
(iii) a third nucleic acid sequence which encodes a signal-dampening component (SDC) comprising a signal-dampening domain (SDD) and a second dimerization domain which specifically binds the first dimerization domain of the membrane-tethering compounds.

36. (canceled)
